(19) United States
(12) Patent Application Publication
(10) Pub. No.: US 2016/0313307 A1
(43) Pub. Date: Oct. 27, 2016

(54) DIAGNOSTIC TEST STRIP FOR ORAL SAMPLES AND METHOD OF USE THEREFORE

(71) Applicants: Ted Titmus, Mission Viejo, CA (US); William Pat Price, Henderson, NV (US)

(72) Inventors: Ted Titmus, Mission Viejo, CA (US); William Pat Price, Henderson, NV (US)

(21) Appl. No.: 14/694,938

(22) Filed: Apr. 23, 2015

(51) Int. Cl. G01N 33/52; G01N 21/64; G01N 33/543; G01N 21/78

(52) U.S. Cl. CPC G01N 33/521; G01N 21/78; G01N 21/6428; G01N 33/54366; G01N 2021/7759

(57) ABSTRACT

Some embodiments provide for a diagnostic test strip having a carrier strip, one or more test pads, and one or more boundary projections which have an opening such that the boundary projections substantially surrounds three sides of each of the one or more test pads. Other embodiments provide for a method of detecting analytes in a patient sample, the method involving contacting an embodiment of a diagnostic test strip with a patient's tongue such that the tongue contacts one or more test pads and reading the results of the analysis from the diagnostic test strip.

### BACKGROUND OF THE INVENTION

1. Field of the Invention

The invention generally relates to diagnostic assay materials. More specifically, the invention relates to diagnostic test strips for testing oral fluid and methods for the use of said diagnostic test strips.

2. Description of the Related Art

Many types of assays have been used to detect the presence of various substances, generally referred to as analytes, in physiological fluids such as urine and blood. These assays often involve antigen-antibody reactions; synthetic conjugates comprising radioactive, enzymatic, fluorescent, or visually observable metal sol tags; and specially designed reactor chambers. In all these assays, there is a receptor; e.g., an antibody or chemical, which is specific for the selected analyte; and a means for detecting the presence, and often the amount, of the analyte. While some tests are designed to make a quantitative determination, in many circumstances all that is required is a qualitative positive/negative indication. However, in some circumstances the analyte of interest is present in the test sample in very small concentrations. Such circumstances require an assay to be very sensitive in order to detect the presence, absence, and/or concentration of the desired analyte. False positives and false negatives for qualitative assays can also be problematic.

Unlike other forms of fluid specimens, the collection of oral fluid, such as saliva, for diagnostic purposes is complicated by many factors. These factors include the low volumes of salivary fluid secreted into the oral cavity, the relatively high viscosity of salivary fluid, and the diverse anatomic dispersion of the salivary glands. Because of these factors, the testing of salivary specimens has not been extensively developed. However, it is known that human saliva carries lymphocytes, plasma cells and immunoglobulins that are directly related to the immunoglobulins found in the blood. In addition, saliva carries immunoglobins that are believed to be peculiar to saliva, for example, the antibody known as secretory IgA. Because of the association between immunoglobulins of the blood and saliva, as well as the occurrence of secretory IgA, antigen-antibody tests may prove useful for conducting diagnostic assays with saliva.

Common techniques for collection of salivary fluid involve the use of capillary tubes, micropipette suctioning, chewing on paraffin or foam, and/or aspiration from the mouth into polypropylene syringes. However, such techniques complicate the collection of salivary specimens and increase the likelihood of user error and/or false negatives. What is needed is a simple, accurate assay that provides trustworthy sample collection and detection of the presence, absence, and/or concentration of one or more analytes present in oral fluid. These and other objects and features of the invention will be apparent from the following description, drawings, and claims.

### SUMMARY OF THE INVENTION

Embodiments described herein are directed to diagnostic test strips, and more specifically to test strips having a carrier strip, one or more test pads, and one or more boundary projections which have an opening such that the boundary projections substantially surrounds three sides of each of the one or more test pads. From this description, in conjunction with other items, the advantages of the invention will become clear and apparent more so based upon the hereinafter descriptions and claims, which are supported by drawings with numbers relating to parts, wherein are described in the following sections containing the relating numbers.

In one aspect of the invention, a diagnostic test strip is provided having a carrier strip, one or more test pads, and one or more boundary projections which have an opening such that the boundary projections substantially surrounds three sides of each of the one or more test pads. Advantageously, the one or more test pads and the one or more boundary projections are opposed on the opposite or same side of the carrier strip. The one or more test pads and the one or more boundary projections may be on the same side of the carrier strip and may be substantially at one end of the carrier strip. The opening in the one or more boundary projections may face toward the end of the carrier strip closest to the one or more test pads. Alternatively, the opening in the one or more boundary projections may face away from the end of the carrier strip closest to the one or more tests pads.

In some aspects of the invention, there are two or more boundary projections on the same side of the carrier strip. Optionally, at least one opening in a boundary projection faces away from the closest end of the carrier strip closest to the one or more tests pads and the opening in another boundary region faces toward the closest end of the carrier strip.

In another aspect, there are two or more boundary projections on the same side of the carrier strip and at least one each of the two or more boundary projections are substantially at the opposite ends of the carrier strip and the opening in each one faces toward the nearest end. Alternatively, the test strip may include two or more boundary projections on the same side of the carrier strip further wherein at least one each of the two or more boundary projections are substantially at the opposite ends of the carrier strip and the opening in each one faces away from the nearest end. In still another embodiment, there are two or more boundary projections on the same side of the carrier strip and further wherein at least one each of the two or more boundary projections are substantially at the opposite ends of the carrier strip and the opening in one faces away from the nearest end and the opening in the other faces toward the nearest end. Optionally, there may be two or more boundary projections on the same side of the carrier strip and further wherein at least two of the two or more boundary projections are placed substantially in the middle of the carrier strip and the opening in each one faces away from each other and toward the nearest end. In another embodiment, there are two or more boundary projections on the same side of the carrier strip and further wherein at least two of the two or more boundary projections are placed substantially in the middle of the carrier strip and the opening in each one faces toward each other. It will be appreciated that there may be two or more boundary projections on the same side of the carrier strip and further wherein at least two of the two or more boundary projections are placed substantially in the middle of the carrier strip and the opening in each one faces the same end of the test strip. In some embodiments, there may be two or more boundary projections on the same side of the carrier strip and further wherein at least one of the two or more boundary projections is placed substantially in the middle of the carrier strip and the other is placed substantially at one end of the test strip and the opening in each one faces away from each other and toward the nearest end. In other embodiments, there are two or more boundary projections on the same side of the carrier strip and further wherein at least one of the two or more boundary projections is placed substantially in the middle of the carrier strip and the other is placed substantially at one end of the test strip and the opening in each one faces toward the same end of the carrier strip. There may be two or more boundary projections on the same side of the carrier strip and further wherein at least one of the two or more boundary projections is placed substantially in the middle of the carrier strip and the other is placed substantially at one end of the test strip and the opening in each one faces toward the same side of the carrier strip. In some embodiments, there are two or more boundary projections on the same side of the carrier strip and further wherein at least one of the two or more boundary projections is placed substantially in the middle of the carrier strip and the other is placed substantially at one end of the test strip and the opening in each one faces substantially toward opposite sides of the carrier strip.

In some embodiments, the sides of the one or more boundary projections slope downward towards the opening of the boundary projection. The sides of the one or more boundary projections may extend substantially for the entire length of the one or more test pads in other aspects of the invention. A diagnostic test strip is also contemplated, wherein the sides of the one or more boundary projections extend partially along the length of the one or more test pads inside and closest to the end the boundary projection.

In one aspect, the diagnostic test strip includes two test pads. Optionally, the test strip comprises one test pad. The one or more test pads may each contain a test reagent. The one or more test pads may each contain the same or a different test reagent. Advantageously, the diagnostic test strip includes one or more test pads that are on the same side of the carrier strip and contain two test reagents on different regions of the test pad. The test reagents may be arranged in a pattern to give a signal to the user.

The carrier strip may be porous or non-porous. Similarly, the one or more test pads may be porous or non-porous.

In certain embodiments, there are at least two or more test pads each with a different test reagent and each reagent tests for a different marker on the same analyte. Optionally, at least one test pad further contains a signaling reagent.

The diagnostic test strip may also include a substantially non-porous handle attached on one end or side of the carrier strip. The carrier strip may be shaped in a number of configurations. It may be substantially square shaped, oval shaped, or substantially circularly shaped.

In another embodiment, there are at least two boundary projections on the same side of the carrier strip and the projections are placed substantially opposite on substantially the edge of the circularly shaped carrier strip. The openings in the boundary projections may face toward the edge of the carrier strip or away from the edge of the carrier strip. Optionally, there may be at least one boundary projection in the substantially center of the carrier strip. Alternatively, there may be at least two boundary projections on the same side of the carrier strip each substantially in the center of the carrier strip and the openings in each one face substantially away from each other. In still another aspect of the invention, there may be three or more boundary projections on the same side of the carrier strip placed substantially at the edge of the carrier strip and substantially evenly spaced from each other. The openings in the boundary projections may face substantially toward the edge of the carrier strip or substantially away from the edge of the carrier strip. The openings in the boundary projections may alternate in facing substantially to and away from the edge of the carrier strip. In some aspects of the invention, the diagnostic test strip includes two or more concentric circles of multiple boundary projections on the same side of the carrier strip substantially evenly spaced from each other.

In some embodiments, the test strip includes two or more boundary projections and at least one is on the opposite side of the others.

A method for detecting one or more analytes in a patient sample is also disclosed. The method includes contacting the test strip described above with a patient's tongue so that the patient's tongue contacts the one or more test pads; and reading the results from the test strip. The method may also include contacting the test strip with one or more signaling reagents so that the one or more reagents contact the one or more test pads. The test strip may be contacted with the top and the sides of the patient's tongue in a substantially back and forth motion from substantially the tip to substantially the back of with the tongue. Optionally, the test strip is contacted with the top and the sides of the patient's tongue in a substantially side-to-side motion along the width of the tongue or the test strip may be contacted with the top and the sides of the patient's tongue in a substantially circular motion.

The one or more test pads and one or more boundary projections may optionally be present on any side of the carrier strip in any desired arrangement. Furthermore, the openings in the boundary projections may be oriented in any arrangement relative to the carrier strip. Consequently, the boundary projections direct the application of samples, such as saliva, to the one or more test pads. Optionally, the one or more test pads contain test reagents and/or signaling reagents that detect the presence, absence, and/or concentration of one or more analytes of interest. Other embodiments provide for a method of detecting analytes in a patient sample, the method involving contacting an embodiment of a diagnostic test strip with a patient's tongue such that the tongue contacts one or more test pads and reading the results of the analysis from the diagnostic test strip.

### BRIEF DESCRIPTION OF THE DRAWINGS

FIG. 1A is a side view of an embodiment of a diagnostic test strip having boundary projections substantially surrounding one test pad with an opening directed substantially toward one end of the test pad.

FIG. 1B is a top view of an embodiment of a diagnostic test strip having boundary projections substantially surrounding one test pad with an opening directed substantially toward one end of the test pad.

FIG. 1C is a side view of an embodiment of a diagnostic test strip having boundary projections substantially surrounding one test pad with an opening directed substantially toward one end of the test pad.

FIG. 1D is a top view of an embodiment of a diagnostic test strip having boundary projections substantially surrounding one test pad with an opening directed substantially toward one end of the test pad.

FIG. 2A is a top view of an embodiment of a diagnostic test strip having boundary projections substantially surrounding two test pads with openings directed substantially towards each test pad.

FIG. 2B is a top view of an embodiment of a diagnostic test strip having boundary projections substantially surrounding two test pads with openings directed substantially away from each test pad.

FIG. 2C is a top view of an embodiment of a diagnostic test strip having boundary projections substantially surrounding two test pads with openings directed substantially away from each test pad.

FIG. 2D is a top view of an embodiment of a diagnostic test strip having boundary projections substantially surrounding two test pads with openings directed substantially towards each test pad.

FIG. 2E is a top view of an embodiment of a diagnostic test strip having boundary projections substantially surrounding two test pads with openings directed substantially towards one side of the diagnostic test strip.

FIG. 3A is a top view of an embodiment of a diagnostic test strip having boundary projections substantially surrounding three test pads with openings directed substantially towards one side of the diagnostic test strip and substantially away from a handle.

FIG. 3B is a top view of an embodiment of a diagnostic test strip having boundary projections substantially surrounding three test pads with openings directed substantially towards one side of the diagnostic test strip and substantially towards a handle.

FIG. 3C is a top view of an embodiment of a diagnostic test strip having boundary projections substantially surrounding three test pads with openings directed substantially away from the cluster of test pads.

FIG. 3D is a top view of an embodiment of a diagnostic test strip having boundary projections substantially surrounding three test pads with openings directed substantially away from the cluster of test pads.

FIG. 3E is a top view of an embodiment of a diagnostic test strip having boundary projections substantially surrounding three test pads with two openings directed substantially towards one side of the test strip and a third opening directed substantially towards the opposite side of the test strip.

FIG. 4A is a top view of an embodiment of a diagnostic test strip having boundary projections substantially surrounding nine test pads with openings directed substantially away from the center of the cluster of test pads.

FIG. 4B is a top view of an embodiment of a diagnostic test strip having boundary projections substantially surrounding nine test pads with openings directed substantially towards the center of the cluster of test pads.

FIG. 5A is a top view of an embodiment of a diagnostic test strip having boundary projections surrounding seven test pads.

FIG. 5B is a perspective view of an embodiment of a diagnostic test strip having boundary projections surrounding seven test pads.

FIG. 6A is a top view of an embodiment of a diagnostic test strip having four test pads, each of which has multiple test pad layers.

FIG. 6B is an expanded view of an embodiment of a test pad having two test pad layers.

FIG. 6C is a perspective view of an embodiment of a test pad having two test pad layers.

FIG. 6D is an expanded view of an embodiment of a test pad having four test pad layers.

FIG. 6E is a perspective view of an embodiment of a test pad having four test pad layers.

FIG. 6F is an expanded view of an embodiment of a test pad having three test pad layers.

FIG. 6G is a perspective view of an embodiment of a test pad having three test pad layers.

FIG. 6H is an expanded view of an embodiment of a test pad having six test pad layers.

FIG. 6I is a perspective view of an embodiment of a test pad having six test pad layers.

### DETAILED DESCRIPTION

The present application relates to U.S. patent application Ser. No. ______, ______ filed entitled “DIAGNOSTIC TEST STRIPS WITH MULTIPLE LAMINATED LAYERS CONTAINING ONE OR MORE REAGENT-CARRYING PADS IN ONE OR MORE LAYERS”, Attorney Docket Number TTUSA.005A2, U.S. patent application Ser. No. ______, ______ filed entitled “MECHANICAL ATTACHMENT OF TEST PADS TO A DIAGNOSTIC TEST STRIP”, Attorney Docket Number TTUSA.006A2, U.S. patent application Ser. No. ______, filed ______ entitled “MECHANICAL ATTACHMENT OF TEST PADS TO A DIAGNOSTIC TEST DEVICE”, Attorney Docket Number TTUSA.007A2, U.S. patent application Ser. No. ______, filed ______ entitled “DIAGNOSTIC TEST STRIP WITH SELF-ATTACHING TEST PADS AND METHODS OF USE THEREFORE”, Attorney Docket Number TTUSA.008A2, U.S. patent application Ser. No. ______, filed ______ entitled “DIAGNOSTIC TEST STRIPS WITH FLASH MEMORY DEVICES AND METHODS OF USE THEREFORE”, Attorney Docket Number TTUSA.009A2, U.S. patent application Ser. No. ______, filed ______ entitled “DIAGNOSTIC TEST STRIPS HAVING ONE OR MORE TEST PAD LAYERS AND METHOD OF USE THEREFORE, Attorney Docket Number TTUSA.011A2, U.S. patent application Ser. No. ______, filed ______ entitled “SINGLE USE MEDICAL TEST PACKAGING”, Attorney Docket Number TTUSA.012A2, U.S. patent application Ser. No. ______, filed ______ entitled “DIAGNOSTIC TEST STRIPS FOR DETECTION OF PAST OR PRESENT INFECTION OF VARIOUS STRAINS OF HEPATITIS” Attorney Docket Number TTUSA.013A2, and U.S. patent application Ser. No. ______, filed ______ entitled “DIAGNOSTIC TEST STRIPS FOR DETECTION OF PRE-SPECIFIED BLOOD ALCOHOL LEVEL” Attorney Docket Number TTUSA.014A2, all of whom have the inventors Ted Titmus and William Pat Price, all of which are filed herewith this even date, all of the disclosures of which are hereby expressly incorporated by reference in their entirety and are hereby expressly made a portion of this application.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. Descriptions of unnecessary parts or elements may be omitted for clarity and conciseness, and like reference numerals refer to like elements throughout. In the drawings, the size and thickness of layers and regions may be exaggerated for clarity and convenience. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Some embodiments of the invention provide for a diagnostic test strip having a carrier strip, one or more test pads, and one or more boundary projections. Moreover, the boundary projections have an opening such that they substantially surround three sides of each of the one or more test pads. The one or more test pads and the one or more boundary projections are optionally opposed on any side of the carrier strip, and specifically on the opposite or same side of the carrier strip. In this regard, the one or more test pads and the one or more boundary projections may be located on any side and/or any end of the carrier strip.

Other embodiments provide a method for detecting one or more analytes in a sample, including but not limited to saliva, having contacting an embodiment of a diagnostic test strip with a patient's tongue so that the patient's tongue contacts the one or more test pads; and results can be read from the diagnostic test strip. These methods include contacting the test strip with one or more signaling reagents so that the one or more reagents contact the one or more test pads. Furthermore, the embodiments may be contacted with the top and the sides of the patient's tongue in a substantially back and forth motion from substantially the tip to substantially the back of with the tongue. Alternatively, embodiments may be contacted with the top and the sides of the patient's tongue in a substantially side-to-side motion along the width of the tongue. Likewise, embodiments may be contacted with the top and the sides of the patient's tongue in a substantially circular motion.

As disclosed below, various features of the embodiments and methods of using the embodiments enable both trained and untrained personnel to reliably detect the presence, absence, and/or concentration of one or more analytes in a sample. Indeed, features of the embodiments and methods for their use allow for the detection of extremely small quantities of one or more particular analytes while avoiding false positives and false negatives. Furthermore, features of the embodiments and methods for their use allow for accurate and trustworthy attainment and/or storage of information related to the tested sample. Optionally, embodiments may both produce a signal that communicates information to the user and store information related to the test sample in one or more memory devices. Consequently, the invention is ideal for use in both prescription and over-the-counter assay test kits which will enable a consumer to self diagnose themselves and others, or test food and/or water prior to consumption.

Any method's results may be read visually by an embodiment's user, if the application so desires, and/or any method's results may be stored in a memory device for recordation and later access. Alternatively, the results may be read by someone other than the user or the supplier of the sample. In some circumstances, the results of the method will be restricted from the user of the embodiment and/or the supplier of the sample analyzed.

Embodiments of the invention can be used to detect any analyte which has heretofore been assayed using known immunoassay procedures, or known to be detectable by such procedures. Furthermore, it is envisioned that known methods can be modified as needed to afford suitable test reagents and/or signaling reagents that will detect analytes that are similar to analytes that have been previously detected using known procedures.

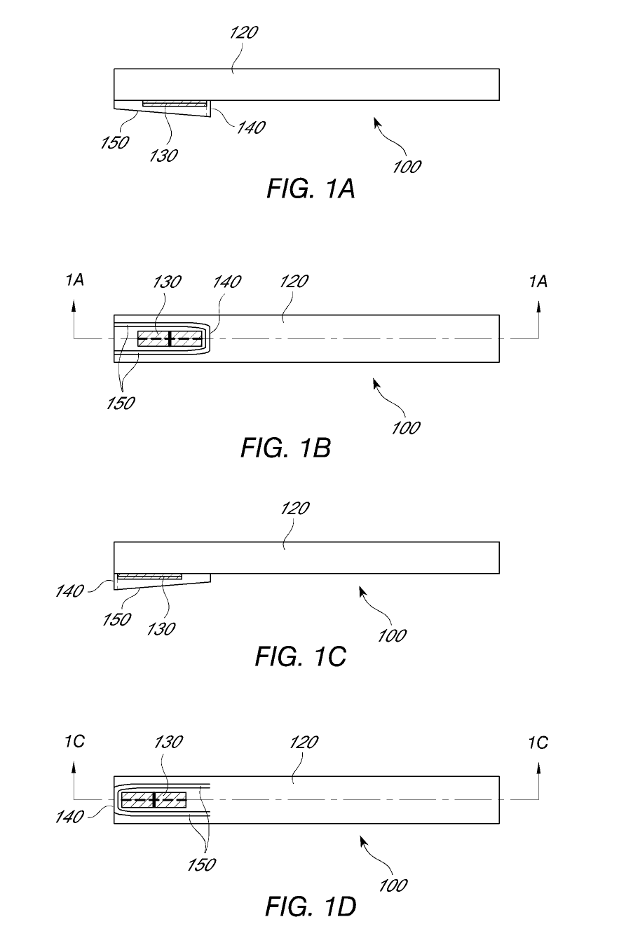

Referring to the drawings, FIG. 1A illustrates schematically a side view of an embodiment of a diagnostic test strip, 100, having a carrier strip, 120, and one test pad, 130, located on carrier strip 120. Optionally, test pad 130 may be recessed into carrier strip 120. The embodiment in FIG. 1A also possesses boundary projections 140 and 150. Boundary projection 140 serves as a backstop in this embodiment while boundary projections 150 serves as sides. Optionally, diagnostic test strip 100 may have a handle. FIG. 1B illustrates schematically a top view of diagnostic strip 100. From FIG. 1B, one can see that test pad 130 is surrounded by boundary projections 150 on two sides and boundary projection 140 on one side such that there is an opening in the boundary projections located substantially towards the end of diagnostic test strip 100. With such an arrangement of boundary projections 140 and 150, this embodiment is suited for scraping a patient's tongue substantially towards the back of the patient's throat. Such a scraping movement will afford collection of a sample of oral fluid within the boundary projections that substantially surround test pad 130.

FIGS. 1C and 1D illustrate schematically an alternative embodiment which has openings in its boundary projections such that the embodiment is suited for placement on a patient's tongue towards the back of the patient's throat and scraping the tongue by pulling the embodiment towards the patient's teeth. FIG. 1C illustrates schematically a side view of an embodiment of a diagnostic test strip, 100, having a carrier strip, 120, and one test pad, 130, located on carrier strip 120. Optionally, test pad 130 may be recessed into carrier strip 120. The embodiment in FIG. 1A also possesses boundary projections 140 and 150. Boundary projection 140 serves as a backstop in this embodiment while boundary projections 150 serves as sides. Optionally, diagnostic test strip 100 may have a handle. FIG. 1D illustrates schematically a top view of diagnostic strip 100. From FIG. 1D, one can see that test pad 130 is surrounded by boundary projections 150 on two sides and boundary projection 140 on one side such that there is an opening in the boundary projections located substantially towards the middle of diagnostic test strip 100 and away from its end. With such an arrangement of boundary projections 140 and 150, a scraping movement will afford collection of a sample of oral fluid within the boundary projections that substantially surround test pad 130.

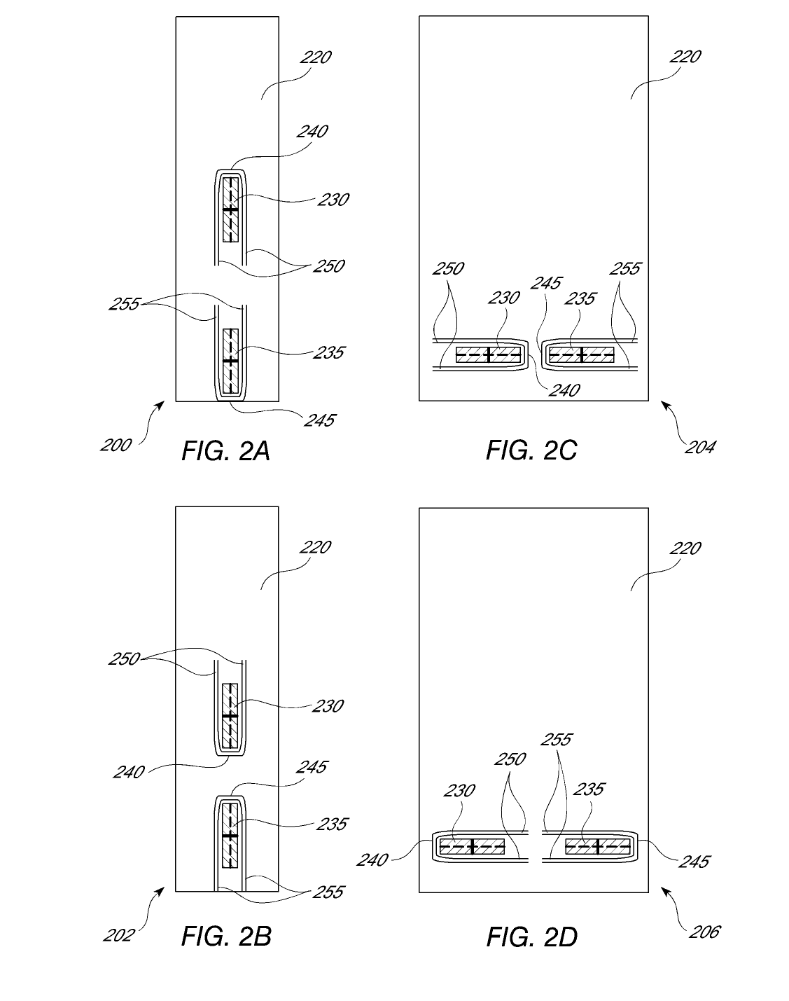

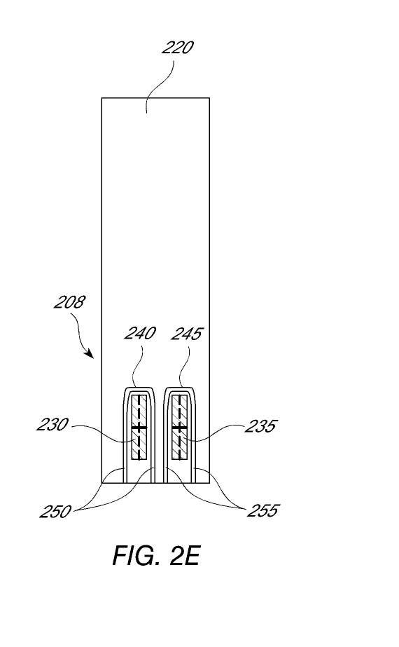

FIGS. 2A and 2B illustrate schematically an alternative embodiment which has openings in its boundary projections such that the embodiment is suited for placement on a patient's tongue and scraping with both backwards and forwards motions to collect oral fluid. FIG. 2A illustrates schematically a top view of an embodiment of a diagnostic test strip, 200, having a carrier strip, 220, and two test pads, 230 and 235, located on carrier strip 220. Optionally, test pads 230 and/or 235 may be recessed into carrier strip 220. The embodiment in FIG. 2A also possesses boundary projections 240, 245, 250, and 255. Boundary projections 240 and 245 serve as backstops in this embodiment while boundary projections 250 and 255 serve as sides. Optionally, diagnostic test strip 200 may have a handle. From FIG. 2A, one can see that test pads 230 and 235 are surrounded by boundary projections 250 and 255 on two sides, respectively, and boundary projections 240 and 245 on one side, respectively, such that there is an opening in the boundary projections surrounding test pad 230 directed substantially towards test pad 235. Similarly, there is an opening in the boundary projections surrounding test pad 235 directed substantially towards test pad 230. With such an arrangement of boundary projections, a back-and-forth scraping movement will afford collection of a sample of oral fluid within the boundary projections that substantially surround test pads 230 and 235.

FIG. 2B illustrates schematically a top view of an alternative embodiment of a diagnostic test strip, 202, having a carrier strip, 220, and two test pads, 230 and 235, located on carrier strip 220. Optionally, test pads 230 and/or 235 may be recessed into carrier strip 220. The embodiment in FIG. 2B also possesses boundary projections 240, 245, 250, and 255. Boundary projections 240 and 245 serve as backstops in this embodiment while boundary projections 250 and 255 serve as sides. Optionally, diagnostic test strip 202 may have a handle. From FIG. 2B, one can see that test pads 230 and 235 are surrounded by boundary projections 250 and 255 on two sides, respectively, and boundary projections 240 and 245 on one side, respectively, such that there is an opening in the boundary projections surrounding test pad 230 directed substantially away from test pad 235. Similarly, there is an opening in the boundary projections surrounding test pad 235 directed substantially away from test pad 230. With such an arrangement of boundary projections, a back-and-forth scraping movement will also afford collection of a sample of oral fluid within the boundary projections that substantially surround test pads 230 and 235.

FIGS. 2C and 2D illustrate schematically an alternative embodiment which has openings in its boundary projections such that the embodiment is suited for placement on a patient's tongue and scraping with side-to-side motions to collect oral fluid. FIG. 2C illustrates schematically a top view of an embodiment of a diagnostic test strip, 204, having a carrier strip, 220, and two test pads, 230 and 235, located on carrier strip 220. Optionally, test pads 230 and/or 235 may be recessed into carrier strip 220. The embodiment in FIG. 2A also possesses boundary projections 240, 245, 250, and 255. Boundary projections 240 and 245 serve as backstops in this embodiment while boundary projections 250 and 255 serve as sides. Optionally, diagnostic test strip 204 may have a handle. From FIG. 2C, one can see that test pads 230 and 235 are surrounded by boundary projections 250 and 255 on two sides, respectively, and boundary projections 240 and 245 on one side, respectively, such that there is an opening in the boundary projections surrounding test pad 230 directed substantially away from test pad 235 and substantially towards one side of the embodiment. Similarly, there is an opening in the boundary projections surrounding test pad 235 directed substantially away from test pad 230 and substantially towards the other side of the embodiment. With such an arrangement of boundary projections, a side-to-side scraping movement will afford collection of a sample of oral fluid within the boundary projections that substantially surround test pads 230 and 235.

FIG. 2D illustrates schematically a top view of an alternative embodiment of a diagnostic test strip, 206, having a carrier strip, 220, and two test pads, 230 and 235, located on carrier strip 220. Optionally, test pads 230 and/or 235 may be recessed into carrier strip 220. The embodiment in FIG. 2D also possesses boundary projections 240, 245, 250, and 255. Boundary projections 240 and 245 serve as backstops in this embodiment while boundary projections 250 and 255 serve as sides. Optionally, diagnostic test strip 206 may have a handle. From FIG. 2D, one can see that test pads 230 and 235 are surrounded by boundary projections 250 and 255 on two sides, respectively, and boundary projections 240 and 245 on one side, respectively, such that there is an opening in the boundary projections surrounding test pad 230 directed substantially towards test pad 235. Similarly, there is an opening in the boundary projections surrounding test pad 235 directed substantially towards test pad 230. With such an arrangement of boundary projections, a side-to-side scraping movement will also afford collection of a sample of oral fluid within the boundary projections that substantially surround test pads 230 and 235.

FIG. 2E illustrates schematically a top view of an embodiment of a diagnostic test strip, 208, having a carrier strip, 220, and two test pads, 230 and 235, located on carrier strip 220. Optionally, test pads 230 and/or 235 may be recessed into carrier strip 220. The embodiment in FIG. 2D also possesses boundary projections 240, 245, 250, and 255. Boundary projections 240 and 245 serve as backstops in this embodiment while boundary projections 250 and 255 serve as sides. Optionally, diagnostic test strip 208 may have a handle. From FIG. 2E, one can see that test pads 230 and 235 are surrounded by boundary projections 250 and 255 on two sides, respectively, and boundary projections 240 and 245 on one side, respectively, such that there is an opening in the boundary projections surrounding test pads 230 and 235 directed substantially towards one end of diagnostic test strip 208. With such an arrangement of boundary projections, a forward scraping movement will afford collection of a sample of oral fluid within the boundary projections that substantially surround test pads 230 and 235. It is also readily envisioned that the openings in the boundary projections may both face any of the three remaining sides to afford embodiments in which the pushing and/or pulling of test strip 208 towards the side with the open boundary projections will afford the collection of a sample of oral fluid within the boundary projections that substantially surround test pads 230 and 235.

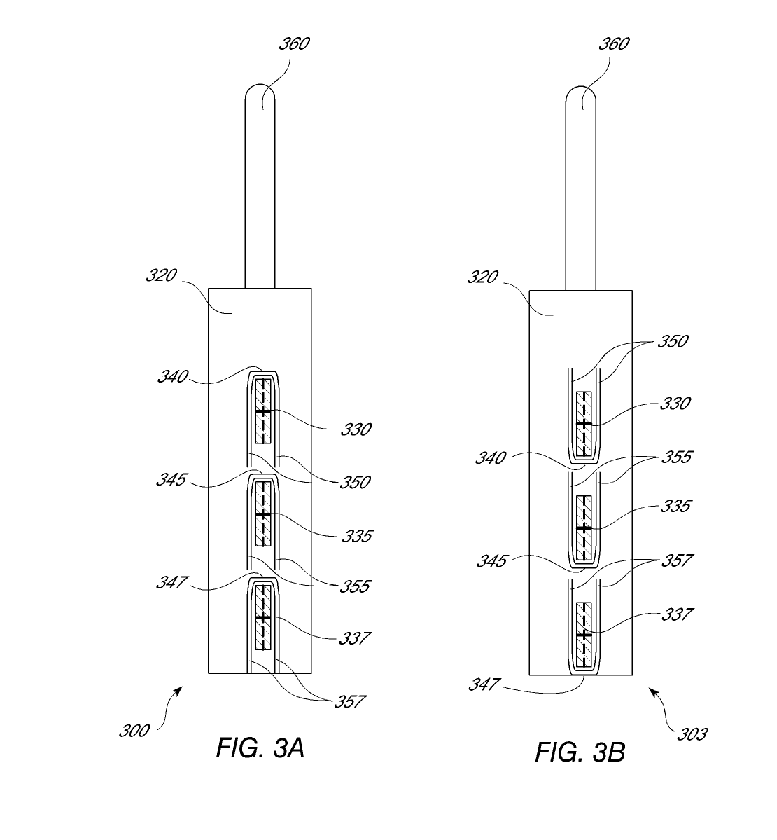

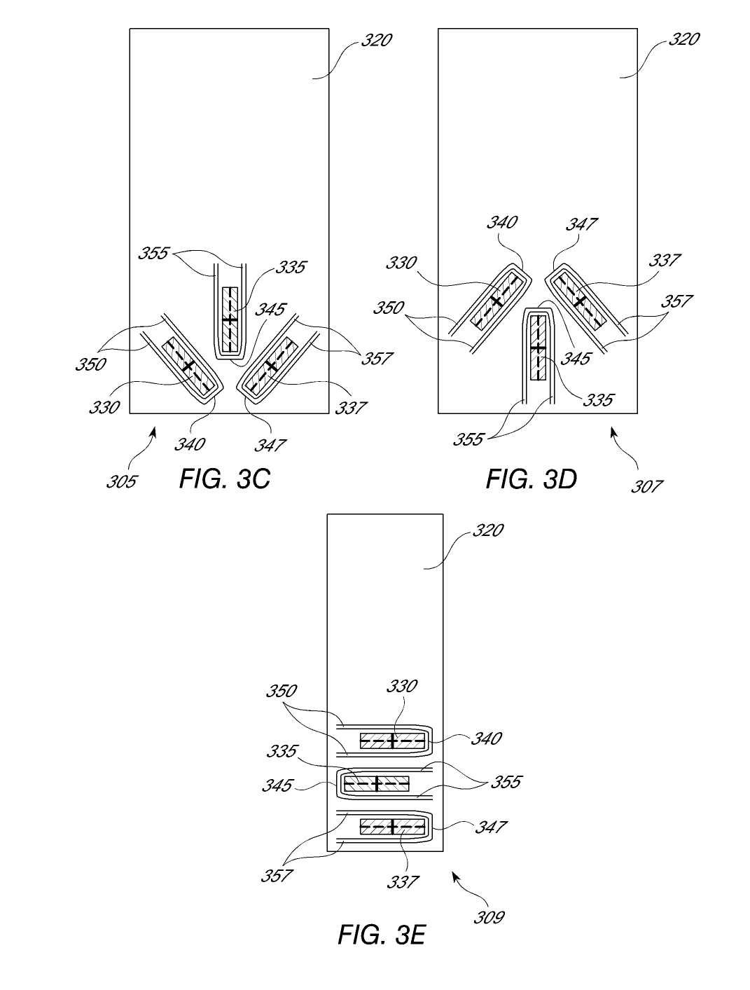

FIG. 3A illustrates schematically an embodiment which has openings in its boundary projections such that the embodiment is suited for placement on a patient's tongue and scraping substantially towards the back of the patient's throat. FIG. 3A illustrates schematically a top view of an embodiment of a diagnostic test strip, 300, having a carrier strip, 320, and three test pads, 330, 335, and 337, located on carrier strip 320. Optionally, test pads 330, 335, and 337 may be recessed into carrier strip 320. The embodiment in FIG. 3B also possesses boundary projections 340, 345, 347, 350, 355, and 357. Boundary projections 340, 345, and 347 serve as backstops in this embodiment while boundary projections 350, 355, and 357 serve as sides. Diagnostic test strip 303 also has an optional handle, 360, in contact with the carrier strip. From FIG. 3A, one can see that test pad 330 is surrounded by boundary projections 350 on two sides and boundary projection 340 on one side such that there is an opening in the boundary projections directed substantially away from handle 360. Similarly, test pad 335 is surrounded by boundary projections 355 on two sides and boundary projection 345 on one side such that there is an opening in the boundary projections directed substantially away from handle 360. Likewise, test pad 337 is surrounded by boundary projections 357 on two sides and boundary projection 347 on one side such that there is an opening in the boundary projections directed substantially away from handle 360. With such an arrangement of boundary projections a scraping movement in the direction substantially away from handle 360 will afford collection of a sample of oral fluid within the boundary projections that substantially surround test pads 330, 335, and 337.

FIG. 3B illustrates schematically an alternative embodiment which has openings in its boundary projections such that the embodiment is suited for placement on a patient's tongue towards the back of the patient's throat and scraping the tongue by pulling the embodiment towards the patient's teeth. FIG. 3B illustrates schematically a top view of an embodiment of a diagnostic test strip, 303, having a carrier strip, 320, and three test pads, 330, 335, and 337, located on carrier strip 320. Optionally, test pads 330, 335, and 337 may be recessed into carrier strip 320. The embodiment in FIG. 3B also possesses boundary projections 340, 345, 347, 350, 355, and 357. Boundary projections 340, 345, and 347 serve as backstops in this embodiment while boundary projections 350, 355, and 357 serve as sides. Diagnostic test strip 303 also has an optional handle, 360, in contact with the carrier strip. From FIG. 3B, one can see that test pad 330 is surrounded by boundary projections 350 on two sides and boundary projection 340 on one side such that there is an opening in the boundary projections directed substantially towards handle 360. Similarly, test pad 335 is surrounded by boundary projections 355 on two sides and boundary projection 345 on one side such that there is an opening in the boundary projections directed substantially towards handle 360. Likewise, test pad 337 is surrounded by boundary projections 357 on two sides and boundary projection 347 on one side such that there is an opening in the boundary projections directed substantially towards handle 360. With such an arrangement of boundary projections a scraping movement in the direction of handle 360 will afford collection of a sample of oral fluid within the boundary projections that substantially surround test pads 330, 335, and 337.

FIGS. 3C and 3D illustrate schematically embodiments which have openings in their boundary projections such that the embodiments are suited for placement on a patient's tongue and scraping in a substantially circular motion. FIG. 3C illustrates schematically a top view of an embodiment of a diagnostic test strip, 305, having a carrier strip, 320, and three test pads, 330, 335, and 337, located on carrier strip 320. Optionally, test pads 330, 335, and 337 may be recessed into carrier strip 320. The embodiment in FIG. 3C also possesses boundary projections 340, 345, 347, 350, 355, and 357. Boundary projections 340, 345, and 347 serve as backstops in this embodiment while boundary projections 350, 355, and 357 serve as sides. Optionally, diagnostic test strip 305 may have a handle. From FIG. 3C, one can see that test pad 330 is surrounded by boundary projections 350 on two sides and boundary projection 340 on one side such that there is an opening in the boundary projections directed substantially away from the cluster of test pads. Similarly, test pad 335 is surrounded by boundary projections 355 on two sides and boundary projection 345 on one side such that there is an opening in the boundary projections directed substantially away from the cluster of test pads. Likewise, test pad 337 is surrounded by boundary projections 357 on two sides and boundary projection 347 on one side such that there is an opening in the boundary projections directed substantially away from the cluster of test pads. With such an arrangement of boundary projections a scraping movement in a direction substantially away from backstops 340, 345, and 347 and in a substantially circular motion will afford collection of a sample of oral fluid within the boundary projections that substantially surround test pads 330, 335, and 337.

FIG. 3D illustrates schematically a top view of an alternative embodiment of a diagnostic test strip, 307, having a carrier strip, 320, and three test pads, 330, 335, and 337, located on carrier strip 320. Optionally, test pads 330, 335, and 337 may be recessed into carrier strip 320. The embodiment in FIG. 3C also possesses boundary projections 340, 345, 347, 350, 355, and 357. Boundary projections 340, 345, and 347 serve as backstops in this embodiment while boundary projections 350, 355, and 357 serve as sides. Optionally, diagnostic test strip 307 may have a handle. From FIG. 3D, one can see that test pad 330 is surrounded by boundary projections 350 on two sides and boundary projection 340 on one side such that there is an opening in the boundary projections directed substantially away from the cluster of test pads. Similarly, test pad 335 is surrounded by boundary projections 355 on two sides and boundary projection 345 on one side such that there is an opening in the boundary projections directed substantially away from the cluster of test pads. Likewise, test pad 337 is surrounded by boundary projections 357 on two sides and boundary projection 347 on one side such that there is an opening in the boundary projections directed substantially away from the cluster of test pads. With such an arrangement of boundary projections a scraping movement in a direction substantially away from backstops 340, 345, and 347 and in a substantially circular motion will afford collection of a sample of oral fluid within the boundary projections that substantially surround test pads 330, 335, and 337.

FIG. 3E illustrates schematically a top view of an alternative embodiment of a diagnostic test strip, 309, having a carrier strip, 320, and three test pads, 330, 335, and 337, located on carrier strip 320. Optionally, test pads 330, 335, and 337 may be recessed into carrier strip 320. The embodiment in FIG. 3E also possesses boundary projections 340, 345, 347, 350, 355, and 357. Boundary projections 340, 345, and 347 serve as backstops in this embodiment while boundary projections 350, 355, and 357 serve as sides. Optionally, diagnostic test strip 309 may have a handle. From FIG. 3E, one can see that test pads 330 and 337 are surrounded by boundary projections 350 and 357, respectively, on two sides and boundary projection 340 and 347, respectively, on one side such that there is an opening in the boundary projections facing substantially the same direction and towards one side of diagnostic test strip 309. Similarly, test pad 335 is surrounded by boundary projections 355 on two sides and boundary projection 345 on one side such that there is an opening in the boundary projections facing substantially the opposite direction of the openings in the boundary projections surrounding test pads 330 and 337. With such an arrangement of boundary projections a scraping movement in a direction substantially side-to-side will afford collection of a sample of oral fluid within the boundary projections that substantially surround test pads 330, 335, and 337.

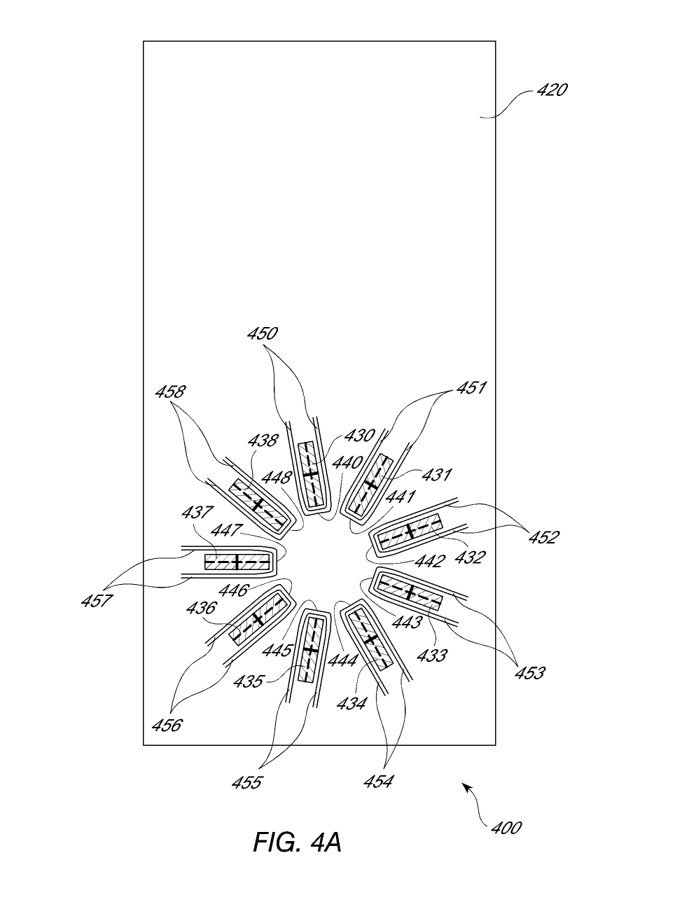

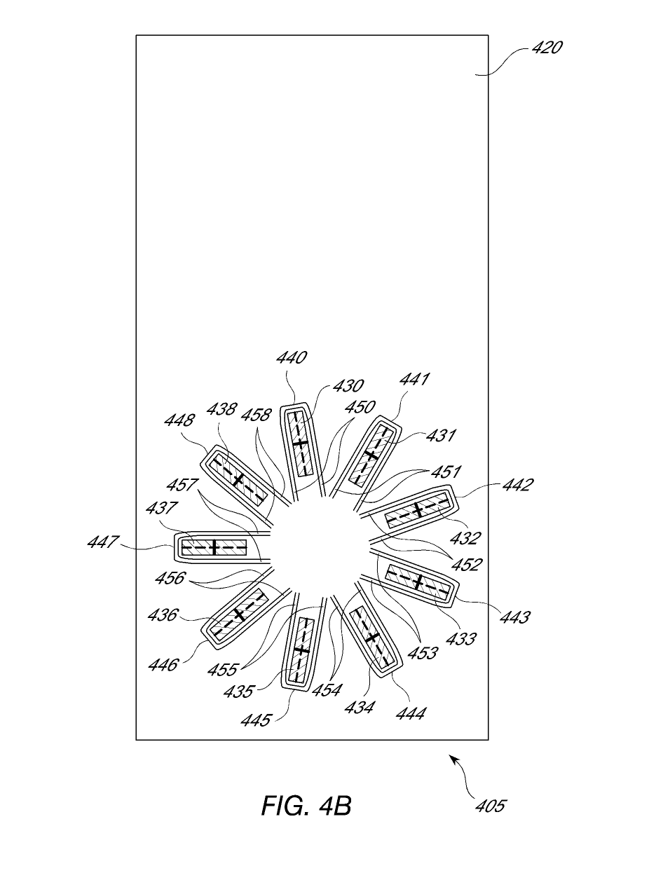

FIGS. 4A and 4B illustrate schematically embodiments which have openings in their boundary projections such that the embodiments are suited for placement on a patient's tongue and scraping in a substantially circular forwards and backwards motion. FIG. 4A illustrates schematically a top view of an embodiment of a diagnostic test strip, 400, having a carrier strip, 420, and nine test pads, 430, 431, 432, 433, 434, 435, 436, 437, and 438, located on carrier strip 420. Optionally, test pads 430, 431, 432, 433, 434, 435, 436, 437, and 438 may be recessed into carrier strip 420. The embodiment in FIG. 4A also possesses backstop boundary projections 440, 441, 442, 443, 444, 445, 446, 447, and 448 and side boundary projections 450, 451, 452, 453, 454, 455, 456, 457, and 458. Optionally, diagnostic test strip 400 may have a handle. From FIG. 4A, one can see that test pad 430 is surrounded by boundary projections 550 on two sides and boundary projection 540 on one side such that there is an opening in the boundary projections directed substantially away from the cluster of test pads. Test pads 431, 432, 433, 434, 435, 436, 437, and 438 have a similar arrangement. With such an arrangement of boundary projections surrounding the test pads, a scraping movement in a direction substantially circular and away from backstops 440, 441, 442, 443, 444, 445, 446, 447, and 448 will afford collection of a sample of oral fluid within the boundary projections that substantially surround test pads 430, 431, 432, 433, 434, 435, 436, 437, and 438.

FIG. 4B illustrates schematically a top view of an alternative embodiment of a diagnostic test strip, 405, having a carrier strip, 420, and nine test pads, 430, 431, 432, 433, 434, 435, 436, 437, and 438, located on carrier strip 420. Optionally, test pads 430, 431, 432, 433, 434, 435, 436, 437, and 438 may be recessed into carrier strip 420. The embodiment in FIG. 4B also possesses backstop boundary projections 440, 441, 442, 443, 444, 445, 446, 447, and 448 and side boundary projections 450, 451, 452, 453, 454, 455, 456, 457, and 458. Optionally, diagnostic test strip 405 may have a handle. From FIG. 4B, one can see that test pad 430 is surrounded by boundary projections 550 on two sides and boundary projection 540 on one side such that there is an opening in the boundary projections directed substantially away from the cluster of test pads. Test pads 431, 432, 433, 434, 435, 436, 437, and 438 have a similar arrangement. With such an arrangement of boundary projections surrounding the test pads, a scraping movement in a direction substantially circular and away from backstops 440, 441, 442, 443, 444, 445, 446, 447, and 448 will afford collection of a sample of oral fluid within the boundary projections that substantially surround test pads 430, 431, 432, 433, 434, 435, 436, 437, and 438.

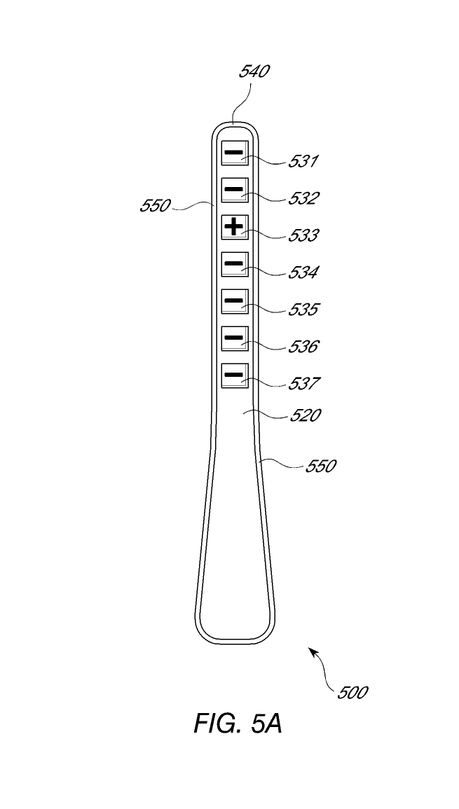

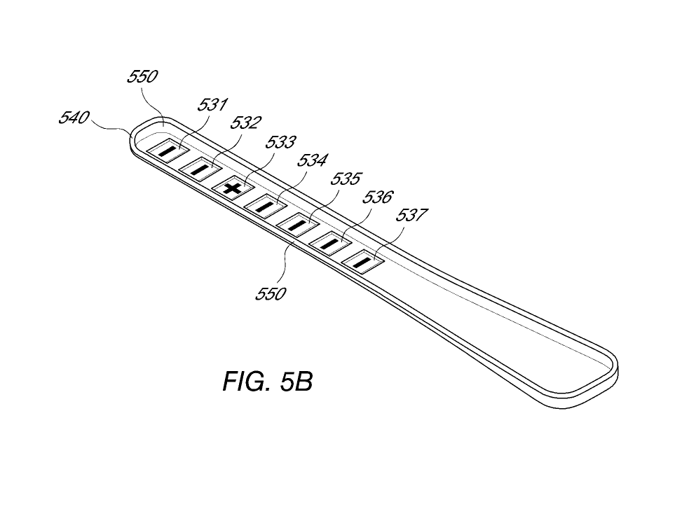

FIG. 5A illustrates schematically a top view of an embodiment of a diagnostic test strip, 500, having a carrier strip 520, and seven test pads, 531, 532, 533, 534, 535, 536, and 537. In FIG. 5A, test pads 531, 532, 534, 535, 536, and 537 all display “minus” signs indicating the absence of an analyte and/or marker of an analyte, while test pad 533 indicates the presence of an analyte and/or marker of an analyte. Diagnostic test strip 500 also possesses boundary projections, with a backstop, 540, and sides 550. FIG. 5B illustrates schematically a perspective view of diagnostic test strip 500.

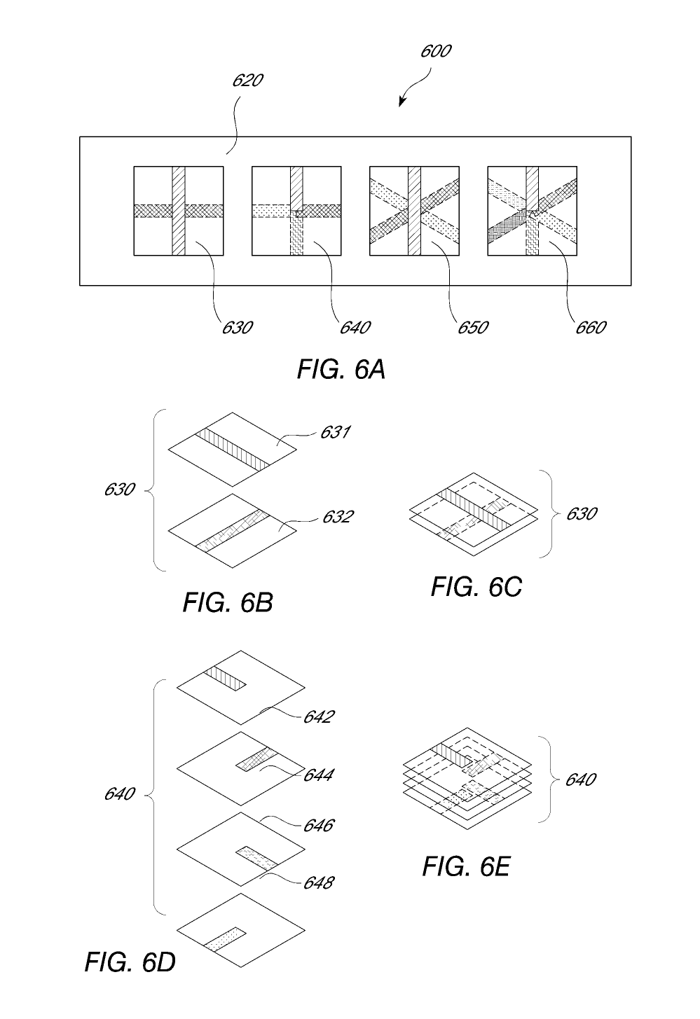

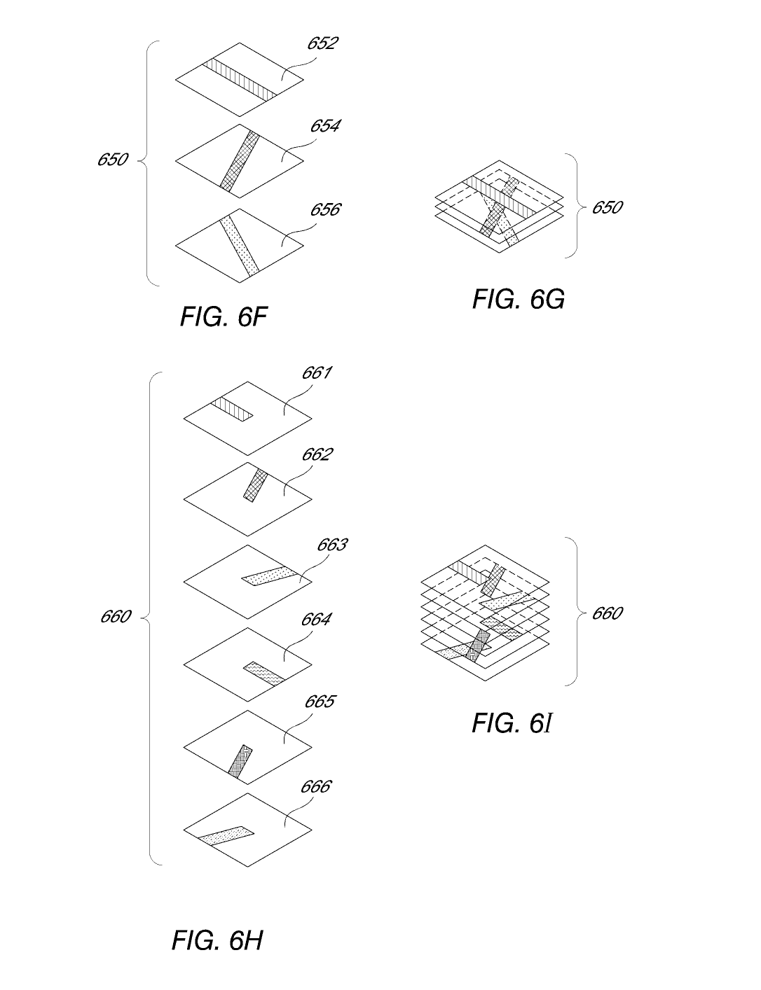

FIG. 6A illustrates schematically a top view of an embodiment of a diagnostic test strip, 600, having a carrier strip, 620, and four test pads, 630, 640, 650, and 660. In FIG. 6A, test pads 630, 640, 650, and 660 are optionally composed of optically transparent multiple layers as illustrated in FIGS. 6B-6I. It is readily envisioned that one or more test pads such as 630, 640, 650, and 660 may be present either alone or in any manner of combination with other test pads, including both single layer test pads and/or multiple layer test pads having any number of test pad layers.

FIG. 6B schematically illustrates an embodiment of a test pad, 630, as comprising two test pad layers, 631 and 632. Each of the test pad layers 631 and 632 are impregnated with test reagent and/or signaling reagent such that the presence of a particular analyte, marker of an analyte, and/or marker of different analytes will result in the generation of a visual signal. In FIG. 6B the visual signal is denoted by a shaded line. Upon detection of the presence of a particular analyte, marker of an analyte, and/or marker of different analytes in each of the test pad layers 631 and 632, a visual signal is generated in each layer that appears to the observer as a “plus” sign. This “plus” sign may confirm the presence of a single analyte and/or multiple analytes, either of which may confirm the diagnosis of one or more diseases, illnesses, or injuries. Thus, the use of test pads having layers comprising optically transparent material impregnated with test reagent and/or signaling reagent provides a user with the ability to perform a more complex analysis. FIG. 6C schematically illustrates the stacking of multiple test pad layers to afford an embodiment of a test pad such as 630.

FIG. 6D schematically illustrates an embodiment of a test pad, 640, as comprising four test pad layers, 642, 644, 646, and 648. Each of the test pad layers 642, 644, 646, and 648 are impregnated with test reagent and/or signaling reagent such that the presence of a particular analyte, marker of an analyte, and/or marker of different analytes will result in the generation of a visual signal. In FIG. 6D the visual signal is denoted by a shaded line. Upon detection of the presence of a particular analyte, marker of an analyte, and/or marker of different analytes in each of the test pad layers 642, 644, 646, and 648, a visual signal is generated in each layer that appears to the observer as a “plus” sign. This “plus” sign may confirm the presence of a single analyte and/or multiple analytes, either of which may confirm the diagnosis of one or more diseases, illnesses, or injuries. Thus, the use of test pads having layers comprising optically transparent material impregnated with test reagent and/or signaling reagent provides a user with the ability to perform a more complex analysis. FIG. 6E schematically illustrates the stacking of multiple test pad layers to afford an embodiment of a test pad such as 640.

FIG. 6F schematically illustrates an embodiment of a test pad, 650, as comprising three test pad layers, 652, 654, and 656. Each of the test pad layers 652, 654, and 656 are impregnated with test reagent and/or signaling reagent such that the presence of a particular analyte, marker of an analyte, and/or marker of different analytes will result in the generation of a visual signal. In FIG. 6F the visual signal is denoted by a shaded line. Upon detection of the presence of a particular analyte, marker of an analyte, and/or marker of different analytes in each of the test pad layers 652, 654, and 656, a visual signal is generated in each layer that appears to the observer as an “asterisk” sign. This “asterisk” sign may confirm the presence of a single analyte and/or multiple analytes, either of which may confirm the diagnosis of one or more diseases, illnesses, or injuries. Thus, the use of test pads having layers comprising optically transparent material impregnated with test reagent and/or signaling reagent provides a user with the ability to perform a more complex analysis. FIG. 6G schematically illustrates the stacking of multiple test pad layers to afford an embodiment of a test pad such as 650.

FIG. 6H schematically illustrates an embodiment of a test pad, 660, as comprising six test pad layers, 661, 662, 663, 664, 665 and 666. Each of the test pad layers 661, 662, 663, 664, 665 and 666 are impregnated with test reagent and/or signaling reagent such that the presence of a particular analyte, marker of an analyte, and/or marker of different analytes will result in the generation of a visual signal. In FIG. 6H the visual signal is denoted by a shaded line. Upon detection of the presence of a particular analyte, marker of an analyte, and/or marker of different analytes in each of the test pad layers 661, 662, 663, 664, 665 and 666, a visual signal is generated in each layer that appears to the observer as an “asterisk” sign. This “asterisk” sign may confirm the presence of a single analyte and/or multiple analytes, either of which may confirm the diagnosis of one or more diseases, illnesses, or injuries. Thus, the use of test pads having layers comprising optically transparent material impregnated with test reagent and/or signaling reagent provides a user with the ability to perform a more complex analysis. FIG. 6I schematically illustrates the stacking of multiple test pad layers to afford an embodiment of a test pad such as 650.

Alternatively, test pads having multiple layers, such as test pads 630, 640, 650, and 660, may indicate the concentration of one or more analytes present in a sample. For example, test pad layers 631, 632, 642, 644, 646, 648, 652, 654, 656, 661, 662, 663, 664, 665, and/or 666 may have different sensitivities to a particular analyte that correlate to a particular concentration of an analyte in a sample. As a non-limiting example represented for test pad 640, at a certain concentration of analyte test pad layer 642 may detect the analyte and generate a signal, such as the shaded line indicated in FIGS. 6D and 6E. At a higher concentration of analyte, both test pad layers 642 and 644 may detect the presence of the analyte and generate a signal, such as the shaded lines indicated in FIGS. 6D and 6E. Still yet, at a higher concentration of analyte, test pad layers 642, 644, and 646 may detect the presence of the analyte and generate a signal, such as the shaded lines indicated in FIGS. 6D and 6E. Finally, at an even higher concentration of analyte, all four test pad layers 642, 644, 646, and 648 may detect the presence of the analyte and generate a signal, such as the shaded lines indicated in FIGS. 6A, 6D, and 6E. Consequently, the different sensitivities of test pad layers 642, 644, 646, and 648 afford information to an observer about the presence and concentration of an analyte in a sample. One will readily appreciate that this illustrative example is capable of extension to any multiple layer test pad comprising any number of test pad layers, including without limitation test pads 630, 650, and/or 660.

One can readily appreciate the application of such embodiments of multiple layer test pads when knowledge of a certain concentration is needed. As a non-limiting application, the detection of a person's blood level alcohol may be achieved using such an embodiment. For a test pad comprising four test pad layers, such as 640, if test pad layer 642 was sensitive to a blood alcohol level of at least 0.02%, test pad layer 644 was sensitive to a blood alcohol level of at least 0.04%, test pad layer 646 was sensitive to a blood alcohol level of at least 0.06%, and test pad layer 648 was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign as in FIGS. 6A, 6D, and 6E would indicate that the sample provider should not legally operate a motor vehicle. One will readily appreciate that this described example is capable of extension to any multiple layer test pad comprising any number of test pad layers, including without limitation test pads 630, 650, and/or 660.

As another non-limiting example, test reagents and/or signaling reagents that are sensitive to markers specific for hepatitis and/or liver damage may be applied to test pads and/or layers within test pads. Consequently, the detection of markers specific for hepatitis and/or liver damage in each test pad and/or layers within test pads would generate a signal. An individual test pad may optionally be sensitive to a single marker for hepatitis and/or liver damage. Alternatively, a single test pad may be sensitive to multiple markers for hepatitis and/or liver damage. In such an embodiment, the detection of one or more markers for hepatitis and/or liver damage may produce a certain signal, e.g. color, indicative of the number of markers detected and/or indicative of the exact marker detected. Alternatively, an embodiment may produce a signal in the form of a shape that indicates the presence of one or more markers indicative of hepatitis and/or liver damage. For example, an embodiment may have a test pad such as 640, with test pad layers 642, 644, 646, and 648 each sensitive to one or more markers specific to an analyte such as viral hepatitis. The respective detection of a marker in each of the test pad layers 642, 644, 646, and 648 would generate a signal such that the detection of a marker in each of the test pad layers 642, 644, 646, and 648 would confirm the diagnosis of a viral hepatitis. Although such an embodiment has been described with specific references to a viral hepatitis, it is envisioned that such an embodiment may readily be tailored to detect any number of analytes and/or markers that are specific to any analyte described below.

### Carrier Strip

The carrier strip provides structural support for the one or more test pads and the one or more boundary projections. As a structural support, many materials suitable for use in preparing the carrier strip are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, wood, cardboard, paper, natural rubber, synthetic rubber, and other suitable polymers. Optionally, the carrier strip may be porous or non-porous. Optionally, the carrier strip may facilitate the transmission of information from the one or more test pads to a memory device. Transmitted information may include, but is not limited to, the presence, absence, and/or concentration of one or more analytes of interest. The carrier strip may facilitate the transmission of information from the one or more test pads to the one or more memory devices by any of several methods known in the art. Such methods include, but are not limited to, the transmission of electrical signals which result from changes in the coulometry, amperometry, or potentiometry of the materials comprising the carrier strip. See U.S. Pat. No. 6,743,635 (Neel et al., issued on Jun. 1, 2001) and U.S. Pat. No. 6,946,299 (Neel at al., issued on Sep. 20, 2005), which are herein incorporated by reference. Alternatively, the carrier strip may facilitate the transmission of optical signals which result from differences in the reflection, transmission, scattering, absorption, fluorescence, or electrochemiluminescense of the materials comprising the carrier strip and/or the test pads. See U.S. Pat. No. 6,040,195 (Carroll et al., issued on Mar. 21, 2000) and U.S. Pat. No. 6,284,550 (Carroll et al., issued on Sep. 4, 2001) which are herein incorporated by reference.

The carrier strip's size and shape is only limited by the desired application of the embodiment. For example, if the desired application is testing a human patient, the embodiment, and consequently the carrier strip, may be smaller or larger depending upon the size of the human patient. Likewise, if the desired application involves testing an animal patient, the embodiment, and consequently the carrier strip, may be smaller or larger depending upon the size of the animal patient. In some embodiments, the carrier strip is about 1, about 1.25, about 1.5, about 1.75, about 2, about 2.25, about 2.5, about 2.75, about 3, about 3.25, about 3.5, about 3.75, about 4, about 1-2, about 1-3, about 1-4, about 2-3, about 2-4, or about 3-4 inches in length. The carrier strip's shape may optionally be varied depending upon the desired application of the embodiment. Some applications may require substantially narrow, fat, rectangular, circular, oval, square, triangular, or other shapes, including combinations of the indicated shapes. It is envisioned that the shape of embodiments can be tailored to the shape of the environment in which the embodiments will be applied. Moreover, the carrier strip may contain boundary projections that substantially surround one, two, three, and/or four sides of one or more test pads to collect and/or direct sample application to the one or more test pads. Furthermore, it is envisioned that a handle may be optionally attached to a carrier strip or in contact with a carrier strip, either directly or indirectly.

### Boundary Projections

The boundary projections facilitate the application of oral liquids such as saliva to the one or more test pads. Substantially surrounding at least two sides, and preferably three sides, of each of the one or more test pads, the boundary projections channel saliva from the mouth to the one or more test pads. Each boundary projection may surround a one or more test pads by extending as a first plane that is substantially parallel to an axis of the embodiment, followed by a second plane extending substantially perpendicular to the first plane, followed by a third plane extending substantially perpendicular to the second plane and parallel to the first plane. Alternatively, each boundary projection may surround a one or more test pads in a substantially “V” shaped manner wherein two planes substantially surround at least two sides, and preferably three sides, of the one or more test pads. Furthermore, it is envisioned that substantially arcuate, substantially oval, and/or substantially circular shaped boundary projections could be utilized or incorporated into the previously described boundary projections.

When embodiments are placed in a patient's mouth and/or pressed against a patient's tongue, the boundary projections scrape and direct saliva onto the one or more tests pads upon movement of the embodiment against the tongue. Optionally, the surface of the boundary projections may contain dimples and/or grooves that impart a textured feel to the tongue.

The opening in the one or more boundary projections may face any direction relative to the carrier strip and the one or more test pads. For example, the openings may face toward the end of the carrier strip closest to the one or more test pads, away from the end of the carrier strip closest to the one or more tests pads, substantially toward the edge of the carrier strip, substantially away from the edge of the carrier strip, or alternate in facing substantially to and away from the edge of the carrier strip. In one embodiment there are two or more boundary projections on the same side of the carrier strip and further wherein at least one opening in a boundary projection faces away from the closest end of the carrier strip closest to the one or more tests pads and the opening in another boundary region faces toward the closest end of the carrier strip. Alternatively, at least one each of the two or more boundary projections may be located at substantially the opposite ends of the carrier strip and the opening in each one may optionally face toward the nearest end, away from the nearest end, or the opening in one boundary may face away from the nearest end and the opening in the other boundary may face toward the nearest end. It is readily envisioned that embodiments may have openings in their boundary projection in such a fashion as to make the embodiment particularly suited for scraping a patient's tongue substantially towards the back of the patient's throat. Alternatively, embodiments may have openings in their boundary projections in such a fashion as to make the embodiment particularly suited for placement on a patient's tongue towards the back of the patient's throat and scraping the tongue by pulling the embodiment towards the patient's teeth. Moreover, embodiments may have openings in their boundary projections in such a fashion as to make the embodiment particularly suited for placement on a patient's tongue and scraping in a side-to-side and/or circular motion.

In additional embodiments, there are at least one boundary projection in the substantially center of the carrier strip. In some embodiments, there are two or more boundary projections on the same side of the carrier strip with at least two of the two or more boundary projections placed substantially in the middle of the carrier strip with their openings optionally facing away from each other and toward the nearest end, toward each other, or toward the same end of the test strip. In another embodiment, there are two or more boundary projections on the same side of the carrier strip where at least one of the two or more boundary projections is placed substantially in the middle of the carrier strip and the other is placed substantially at one end of the test strip and the opening in each one optionally faces away from each other and toward the nearest end, toward the same end, toward the same side, or toward opposite sides of the carrier strip.

In some embodiments, there are three or more boundary projections on the same side of the carrier strip placed substantially at the edge of the carrier strip and substantially evenly spaced from each other. In additional embodiments, there are two or more concentric circles of multiple boundary projections on the same side of the carrier strip substantially evenly spaced from each other. In some embodiments, there are two or more boundary projections and at least one is on the opposite side of the others.

In other embodiments, the sides of the one or more boundary projections generally slope downward towards the opening of the boundary projection. The degree of slope relative to the carrier strip may optionally be about 5, about 10, about 15, about 20, about 25, about 30, about 35, about 40, about 45, about 50, about 55, about 60, about 65, about 70, about 75, about 80, about 85, about 5-15, about 10-20, about 15-25, about 20-30, about 25-35, about 30-40, about 35-45, about 40-50, about 45-55, about 50-60, about 55-65, about 60-70, about 65-75, about 70-80, about 75-85, or about 80-90 degrees. Alternatively, the boundary projections may generally slope downward towards the opening of the projection in a stepwise manner, such that there is an extension of the boundary projection substantially parallel to the carrier strip, followed by a lowering in height of the boundary projection at an angle substantially perpendicular to the carrier strip. Such a stepwise sloping can be repeated as needed to afford a substantially downward slope.

Moreover, the boundary projections may extend substantially perpendicular to the carrier strip for a desired distance, and then the boundary projections may generally slope towards the opening in the boundary projections in any of the above described manners. Alternatively, the boundary projections may extend at substantially non-perpendicular angles to the carrier strip. Such angles include, but are not limited to, about 5, about 10, about 15, about 20, about 25, about 30, about 35, about 40, about 45, about 50, about 55, about 60, about 65, about 70, about 75, about 80, about 85, about 5-15, about 10-20, about 15-25, about 20-30, about 25-35, about 30-40, about 35-45, about 40-50, about 45-55, about 50-60, about 55-65, about 60-70, about 65-75, about 70-80, about 75-85, or about 80-90 degrees. In another embodiment, the sides of the one or more boundary projections may extend substantially for the entire length of the one or more test pads. Alternatively, the sides of the one or more boundary projections extend partially along the length of the one or more test pads inside and closest to the end the boundary projection. In one embodiment, there are at least two boundary projections on the same side of the carrier strip and the projections are placed substantially opposite on substantially the edge of a circularly shaped carrier strip.

The boundary projections may be made of any suitable material known in the art, including but not limited to polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, natural rubber, synthetic rubber, and other suitable polymers. Optionally, the boundary projections may comprise porous or non-porous materials.

### Test Reagents and Signaling Reagents

Test reagents and signaling reagents suitable for inclusion in embodiments are well known in the art. Such reagents include, but are not limited to, polyclonal antisera and monoclonal antibodies that have specific binding properties and high affinity for virtually any antigenic substance. Literature affords many means of preparing such reagents. See, e.g., Laboratory Techniques in Biochemistry and Molecular Biology, Tijssen, Vol. 15, Practice and Theory of Enzyme Immunoassays, chapter 13, The immobilization of Immunoreactants on Solid Phases, pp. 297-328, and the references cited therein which are herein incorporated by reference. Additional assay protocols, reagents, and analytes useful in the practice of the invention are known per se. See, e.g., U.S. Pat. No. 4,313,734 (Leuvering, issued on Feb. 2, 1982), columns 4-18, and U.S. Pat. No. 4,366,241 (Tom et al., issued on Dec. 28, 1982), columns 5-40 which are herein incorporated by reference.

Metal sols, including but not limited to gold sol, and other types of colored particles, including but not limited to, organic dye sols and colored latex particles, that are useful as marker substances in immunoassay procedures are also known per se and suitable for use as test reagents and/or signaling reagents. See, for example, U.S. Pat. No. 4,313,734 (Leuvering, issued on Feb. 2, 1982), the disclosure of which is incorporated herein by reference. For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973) which are herein incorporated by reference.

Test reagents for inclusion in the embodiments may signal directly, such as with an electrical or optical signal (visible either to the naked eye, or with an optical filter or upon applied stimulation to promote fluorescence or phosphorescence). Test reagents may also signal indirectly such as with enzymes, e.g. alkaline phosphatase and/or horseradish peroxidase, in combination with signaling reagents in the form of enzymatic substrates that will generate a signal upon interaction with the enzyme. In some embodiments, the signaling reagent and/or test reagent is incorporated into the test pad. In other embodiments, the signaling reagent and/or test reagent is added to the test sample before application to the test pad. In additional embodiments, the signaling reagent and/or test reagent is added to the test pad after introduction of the test sample.

Alcohol sensitive test reagents and methods are well known in the art. See, e.g. U.S. Pat. No. 5,563,073 (Titmas, issued on Oct. 8, 1996) and Jai Moo Shin et al., Simple Diagnostic Tests to Detect Toxic Alcohol Intoxications, NIH (October 2008), which are hereby incorporated by reference in their entirety. In some embodiments, the test reagent and/or signaling reagent from Alco Screen™ pads, manufactured by Chematics, Inc. located in North Webster, Ind., is incorporated. Optionally, the test reagent and/or signaling reagent from Alco Screen™ pads is incorporated in the one or more test pads, but it may also be applied to the test pad after sample application or it may be applied to the sample before application to the test pad. In some embodiments the test reagent and/or signaling reagent from the alcohol dehydrogenase method (ADH method) is incorporated in the one or more test pads, but it may also be applied to the test pad after sample application or it may be applied to the sample before application to the test pad. In some embodiments the test reagent and/or signaling reagent from the alcohol oxidase method method (ALOx method) is incorporated in the one or more test pads, but it may also be applied to the test pad after sample application or it may be applied to the sample before application to the test pad. In some embodiments the test reagent and/or signaling reagent from the sodium periodate method is incorporated in the one or more test pads, but it may also be applied to the test pad after sample application or it may be applied to the sample before application to the test pad. In some embodiments the test reagent and/or signaling reagent from the potassium permanganate method (PA method) is incorporated in the one or more test pads, but it may also be applied to the test pad after sample application or it may be applied to the sample before application to the test pad.

Test reagents and/or signaling reagents may also detect the storage and handling of embodiments. In some embodiments, test reagents and/or signaling reagents may be sensitive to temperature and if the temperature of the embodiment's environment has exceeded or fallen below a predetermined temperature, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

In some embodiments, test reagents and/or signaling reagents may be sensitive to moisture, and if the humidity of the embodiment's environment has exceeded or fallen below a predetermined level, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

Test reagents and/or signaling reagents may also detect whether a sufficient amount of sample has been applied to an embodiment for analysis. For example, when the sample is saliva, a test reagent and/or signaling reagent specific for a salivary enzyme, such as amylase, may detect the salivary enzyme's presence if a sufficient volume of sample has been applied. The detection of a sufficient sample may optionally be signaled to the user in the form of a color or symbol. Using such embodiments, the user would then know if a sufficient quantity of sample was applied to the one or more test pads to afford an accurate analysis.

Embodiments that detect storage and/or sufficient application of sample volume are particularly capable of reducing the occurrence of false negatives. For example, poor storage conditions may inactivate a test reagent in a test pad. Upon application of sample to such a test pad, no signal may result and a user could believe that an analyte is not present—a false negative. Alternatively, test pads having a pre-printed negative signal may suffer a similar occurrence of a false negative if the test reagent is inactivated because an analytes presence in a sample would not convert the pre-printed negative signal into a positive signal. Likewise, an insufficient volume of sample may generate no signal or a negative signal and cause a user to believe that an analyte is not present.

Any enzyme, antibody, dye buffer, chemical, sol, or combinations thereof may be incorporated so long as the enzyme, antibody, dye buffer, chemical, metal sol, or combinations thereof are capable of detecting the presence of one or more analytes in a sample. See, e.g., U.S. Pat. No. 6,383,736 (Titmas, issued on May 7, 2002), U.S. Pat. No. 7,858,756 (Owens et al., issued on Dec. 28, 2010), and U.S. Pat. No. 7,790,400 (Jehanli et al., issued on Sep. 7, 2010) which are hereby incorporated by reference in their entirety.

### Test Pads

The one or more test pads may be prepared from any bibulous, porous, fibrous, or sorbent material capable of rapidly absorbing a sample. Porous plastics material, such as polypropylene, polyethylene, polyvinylidene flouride, ethylene vinylacetate, acrylonitrile and polytetrafluoroethylene can be used. Optionally, the one or more test pads can be pre-treated with a surface-active agent to reduce any inherent hydrophobicity in the one or more test pads and enhance their ability to absorb a sample. The one or more test pads can also be made from paper or other cellulosic materials, including but not limited to nitrocellulose. Materials that are now used in the nibs of fiber-tipped pens are also suitable for incorporation in the one or more test pads.

Optionally, the one or more test pads may be prepared from non-porous materials. In such circumstances, the test reagents and/or signaling reagents may be coated on the outer surface of the one or more test pads such that contact with a sample containing an analyte will result in the generation of a signal.

Using known methods, test pads may be shaped or extruded in a variety of lengths and cross-sections. Embodiments may possess one or more test pads of various sizes and shapes, and the size and shape of the one or more test pads are only limited by their number, size, and desired application of the embodiment in which they are incorporated within. In some embodiments, the one or more test pads are substantially similar in size and/or shape. In other embodiments, the one or more test pads may differ substantially in size and/or shape. It is readily envisioned that embodiments may possess about one or more test pads, about two or more test pads, about three or more test pads, about four or more test pads, about five or more test pads, about six or more test pads, about seven or more test pads, about eight or more test pads, about nine or more test pads, about ten or more test pads, about 1-4 test pads, about 1-10 test pads about 1-100 test pads, about 2-100 test pads, about 3-100 test pads, about 4-100 test pads, about 5-100 test pads, about 5-75 test pads, about 10-50 test pads, about 15-25 test pads, and individual numbers of test pads therein. The one or more test pads may be made of the same material, or optionally they may be made of different materials or even combinations of different materials.

In some embodiments, test pads may be prepared from a single layer of material. In other embodiments, test pads may be prepared from multiple layers of material. In both circumstances, test reagents and/or signaling reagents may be impregnated in a single layer of material or in multiple layers of material. The impregnation may take any suitable form, including, but not limited to, a substantially uniform impregnation or impregnation with dots or stripes. Test reagents and/or signaling reagents can be impregnated in various concentrations in one or more of the multiple layers to tailor the sensitivity of the test pads to certain analytes. Such sensitivity could afford information about the concentration of an analyte in the sample. Furthermore, the impregnation may optionally be conducted in a manner that will generate a signal observable by the user upon application of a sufficient quantity of sample, detection of an analyte, or proper/improper storage of the embodiment.

When one or more test pads are comprised of multiple layers of material, one or more layers of material may be impregnated with an inert chemical such that a line or “minus sign” is displayed to the user. In some embodiments, the line or “minus sign” could be in the form of a material covering the one or more test pads to give a visual impression of a line or “minus sign” on the one or more test pads. One or more additional layers of the material comprising the one or more test pads could then be impregnated with a test reagent and/or a signaling reagent that upon detecting a sufficient quantity of sample, appropriate storage temperature, and/or the presence of an analyte, the impregnated test reagent and/or signaling reagent will create a perpendicular line such that a “plus sign” will be signaled to the user. In other embodiments, the line or “minus sign” displayed in the one or more test pads could be obscured by color or opaqueness when a test reagent and/or a signaling reagent detects a sufficient quantity of sample, appropriate or inappropriate storage temperature, and/or the presence of an analyte. Alternatively, the individual layers in a test pad may be positioned such that the detection of an analyte in a lower layer of material is obscured by the detection of an analyte in a layer of material positioned above the lower layer. It is also envisioned that embodiments may have arrangements of test pads and/or arrangements of layers within multiple layered test pads such that the detection of an analyte in the test pads or the layers of a test pad generate a signal, such as a “plus sign” or “minus sign” to the user. Such embodiments may comprise at least two layers of material, each capable of generating a line upon detecting an analyte or a certain concentration of an analyte. Optionally, the lines may intersect to generate a “plus” sign or other signal upon the detection of an analyte in the at least two layers of material. Alternatively, embodiments may comprise at least four layers of material, each capable of generating a line upon detecting an analyte or a certain concentration of an analyte in the at least four layers of material. Optionally, the lines may intersect at one or more points such that a “plus” sign or other symbol is formed.

While the aforementioned embodiments have been discussed with reference to “minus” and “plus” signs, it is envisioned that any symbol, including color changes, could be used to convey similar information to a user. The meaning of any desired symbol or color change could be included in the packaging of an embodiment or imprinted on an embodiment.

The test reagents applied to each layer of material may optionally be the same or different. When different test reagents are applied to different layers of material comprising the one or more test pads, the test pad may be tailored to generate a signal indicating the diagnosis of one or more illnesses, diseases, or injuries. One method for achieving such a diagnosis would be to have the individual layers comprising the test pad generate a signal in response to one or more symptoms of one or more illnesses, diseases, or injuries. For example, if the diagnosis of one or more illnesses, diseases, or injuries required the determination of multiple analytes, then the detection of each analyte could produce a portion of a symbol that is visible to the user. Upon formation of a complete symbol, the embodiment would confirm the presence of a certain illness, disease, or injury. Optionally, information relating to each specific analyte could be transferred to the one or more memory devices.

Embodiments may optionally possess one or more test pads and test reagents that detect analytes important to a certain age population (e.g. infants, children, young adults, adults, or elderly individuals). It is also envisioned that embodiments could possess one or more test pads and test reagents that detect analytes important to certain categories of individuals (e.g., law enforcement agents, government employers, military members, chronic drug users, physicians, veterinarians, dentists, parents, private sector employers, aid workers, inmates, hospital patients, nursing home patients, outdoorsmen, immuno-compromised individuals, or students). Embodiments may also be directed to analytes important to geographic regions (e.g. third-world countries, developed countries, or specific climate regions). Such embodiments of the invention simplify the number of different embodiments that a user must purchase or travel with because users can select embodiments that will detect the analytes the users are most interested in, or are most pertinent to a user's current or impending circumstances.

In one embodiment, a single test pad contains or has applied to it a single test reagent and/or signaling reagent suitable for detecting a single analyte. In another embodiment, two or more test pads contain or have applied to one or more of them a single test reagent and/or signaling reagent suitable for detecting a single analyte. Optionally, the single test reagent and/or signaling reagent on or applied to the two or more test pads may be the same or different. Furthermore, when different test reagents and/or signaling reagents are used, the test reagents may be sensitive to the same marker on an analyte or the test reagents may be sensitive to different markers on an analyte. The analyte may optionally be the same or different. When different analytes and different test reagents and/or signaling reagents are used, the analytes and test reagent and/or signaling reagents may be tailored to detect different symptoms of the same illness, disease, or injury. In some embodiments, a diagnosis can be made based upon the detection of all the symptoms specific to an illness, disease, or injury. In other embodiments, a diagnosis can be made based upon the absence of one or more analytes specific to an illness, disease, or injury. Using these described test pads, it is readily apparent that the reduction of false negatives and false positives can be achieved by including redundancy in the embodiments.

In one embodiment, a single test pad may contain or have applied to it two or more reagents suitable for detecting and/or signaling a single analyte. These two or more test reagents and/or signaling reagents may be sensitive to the same marker of an analyte. Optionally, these two or more reagents may be sensitive to different markers on the same analyte. In some embodiments, the two or more test reagents and/or signaling reagents may be applied to the same region of the test pad. In other embodiments, the two or more test reagents and/or signaling reagents may be applied to different regions of the same test pad. The number of test reagents and/or signaling reagents suitable for incorporation or application to a single test pad is limited only by the application of the diagnostic test strip. It is readily envisioned that embodiments may possess about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test reagents and/or signaling reagents incorporated or applied to one or more test pads. Using these described test pads, it is readily apparent that the reduction of false negatives and false positives can be achieved by including redundancy in the embodiments.

The one or more test pads suitable for use in an embodiment will readily detect analytes present in liquid samples, such as saliva. It is also envisioned that a test pad may be capable of detecting an analyte present in solid and/or semi-solid samples. When solid and/or semi-solid samples are analyzed, it is understood that a liquid may optionally be applied to the test pad to facilitate analysis.

When liquids and/or liquid samples are applied to test pads, lateral flow through material may result from surface tension, cohesion, adhesion, wicking, and/or capillary action. In some embodiments, lateral flow is confined to the test pad region. In other embodiments, lateral flow is confined to individual test pads. In further embodiments, lateral flow is confined to individual layers of a multi-layer test pad. The phrase “lateral flow is confined” means that an embodiment does not utilize fluid communication outside of the indicated portion of the embodiment.

### Analytes

An assay based on the principles described herein can be used to determine a wide variety of analytes by choice of appropriate test reagents and/or signaling reagents. The embodiments described herein can be used to test for the existence of analytes including, but not limited to, drugs, especially drugs of abuse, heavy metals, pesticides, pollutants, proteins, polynucleotides such as DNA, RNA, rRNA, tRNA, mRNA, and siRNA, hormones, vitamins, microorganisms such as bacteria, fungi, algae, protozoa, multi-cellular parasites, and viruses, tumor markers, liver function markers, kidney function markers, blood coagulation factors, and toxins. The embodiments may also optionally detect metabolites of each of the aforementioned examples of analytes. Furthermore, some embodiments may also detect their storage conditions, specifically the temperature and humidity of their environment, and/or the application of an appropriate quantity of sample for analysis.

More specific examples of drug analytes include benzheterocyclics, the heterocyclic rings being azepines, diazepines and phenothiazines. Examples of azepines include fenoldopam. Examples of benzodiazepines include alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine. Examples of other benzheterocyclics include, but are not limited to, carbamazepine and imipramine.

Additional drug analytes include alkaloids, such as agents that interact with opioid receptors including morphine, dihydromorphine, desomorphine, hydromorphone, nicomorphine, oxymorphone, hydromorphinol, nalbuphine, naloxone, naltrexone, buprenorphine, etorphine, metopon, diacetyldihydromorphine, thebacon, methodone, codeine, hydrocodone, dihydrocodeine, oxycodone, papaveretum, oripavine, thebaine, tapentadol, and heroin; agents that exert effects on serotonin receptors, such as cocaine (and other reuptake inhibitors, including norepinephrine, dopamine, and serotonin reuptake inhibitors); cocaine metabolites such as benzoylecgonine; ergot alkaloids; steroid alkaloids; iminazoyl alkaloids; quinazoline alkaloids; isoquinoline alkaloids; quinoline alkaloids; and diterpene alkaloids.

The next group of drug analyte includes steroids, including the estrogens, gestogens, androgens, andrenocortical steroids, bile acids, cardiotonic glycosides and aglycones, which includes digoxin and digoxigenin, saponins and sapogenins, their derivatives and metabolites.

The next group of drug analytes is the barbiturates, such as barbital, allobarbital, amobarbital, aprobarbital, alphenal, brallobarbital, Phenobarbital, pentobarbital, Nembutal, secobarbital, diphenylhydantonin, primidone, and ethosuximide. Additionally, drugs similar in effect to barbiturates are potential analytes, such as methaqualone, cloroqualone, diproqualone, etaqualone, mebroqualone, mecloqualone, methylmethaqualone, and nitromethaqualone.

The next group of drug analytes is aminoalkylbenzenes, including the phenethylamines such as amphetamine, methamphetamine, lisdexamfetamine, mescaline, and catecholamines, which includes ephedrine, L-dopa, epinephrine, narceine, and papaverine.

The next group of drug analytes includes those derived from marijuana, which includes cannabinol, tetrahydrocannabinol, 11-nor-9-carboxy-delta-9-tetrahydrocannabinol, nabilone, dronabinol, marinol, and cannabinoids such as cannabidiol, cannabinol, and tetrahydrocannabivarin.

The next group of drug analytes are those that interact with the N-methyl d-aspartate (“NMDA”) receptor, including agonists, modulators, and antagonists such as 1-(1-phylcyclohexyl)piperidine (phencyclidine or “PCP”), R-2-amino-5-phosphonopentanoate, 2-amino-7-phosphonoheptanoic acid, (3-[(R)-2-carboxypiperazin-4-yl]-prop-2-enyl-1-phosphonic acid), PEAQX, selfotel, amantadine, dextrallorphan, dextromethorphan, dextrorphan, dizocilpine, ethanol, eticyclidine, gacyclidine, ibogaine, ketamine, memantine, methoxetamine, rolicyclidine, tenocyclidine, tiletamine, neramexane, eliprodil, etoxadrol, dexoxadrol, NEFA, remacemide, delucemine, 8A-PDHQ, aptiganel, HU-211, remacemide, atomoxetine, rhynchophylline, 1-aminocyclopropanecarboxylic acid, 7-chlorokynurenate, 5,7-dichlorokynurenic acid, kynurenic acid, and lacosamide.

The next group of drugs is antibiotics, which include, for example, beta-lactam antiobiotics such as penicillins and cephalosporins, penems and carbapenems, antimicrobials such as aminoglycosides, ansamycins, carbacephems, glycopeptides, lincosamides, lipopetides, macrolides, monobactams, nitrofurans, quionolones, polypeptide-based antibiotics, chloromycetin, actinomycetin, spectinomycin, sulphonamides, trimethoprim, tetracyclines, and beta-lactamase inhibitors such as calvulanic acid, tazobactam, and sulbactam.

Other individual miscellaneous drug analytes include nicotine, caffeine, gamma-hydroxybutyric acid, dextromoramide, ketobemidone, piritramide, dipipanone, phenadoxone, benzylmorphine, nicocodeine, dihydrocodeinone enol acetate, tilidine, meptazinol, propiram, acetyldihydrocodeine, pholcodine, 3,4-methylenedioxymethamphetamine, psilocybin, 5-methoxy-N,N-diisopropyltryptamine, peyote, 2,5-dimethoxy-4-methylamphetamine, 2C-T-7 (a psychotropic entheogen), 2C-B, cathinone, alpha-methyltryptamine, bufotenin, benzylpiperazine, methylphenidate, dexmethylphenidate, laudanum, fentanyl, mixed amphetamine salts (i.e. Adderall), lisdexamfetamine, dextroamphetamine, dextromethamphetamine, pethidine, anabolic steroids, talbutal, butalbital, buprenorphine, xyrem, paregoric, modafinil, difenoxin, diphenoxylate, promethazine, pregabaline, pyrovalerone, atropine, and other Schedule I-V classified drugs, glucose, cholesterol, bile acids, fructosamine, carbohydrates, metals which includes, but is not limited to lead and arsenic, alcohols (i.e. methanol, ethanol, propanol, butanol, and C₅₋₁₀ containing alcohols), meprobamate, serotonin, meperidine, amitriptyline, nortriptyline, lidocaine, procaineamide, acetylprocainearnide, propranolol, griseofulvin, valproic acid, butyrophenones, antihistamines, and anticholinergic drugs, such as atropine.

Pesticide analytes of interest include categories such as algicides, avicides, bactericides, fungicides, herbicides, insecticides, miticides, molluscicides, nematicides, rodenticides, virucides, and specifically polyhalogenated biphenyls, phosphate esters, thiophosphates, carbamates, and polyhalogenated sulfenamides.

Additional chemical analytes of interest include fertilizers such as ammonium derivatives, nitrates, and phosphates; heavy metals such as lead, mercury, uranium, plutonium, arsenic, cadmium, chromium, and nickel

More specific examples of protein analytes include antibodies, protamines, histones, albumins, globulins, scleroproteins, phosphoproteins, mucoproteins, chromoproteins, lipoproteins, nucleoproteins, glycoproteins, proteoglycans, and unclassified proteins, such as somatotropin, prolactin, insulin, and pepsin. A number of proteins found in the human plasma are important clinically and include prealbumin, albumin, α₁-lipoprotein, α₁-acid glycoprotein, α₁-antitrypsin, α₁-glycoprotein, transcortin, 4.6S-postalbumin, tryptophan-poor, α₁-glycoprotein, α₁X-glycoprotein, thyroxin-binding globulin, inter-α-trypsin-inhibitor, Gc-globulin (Gc I-I, Gc 2-1, Gc 2-2), haptoglobin, ceruloplasmin, cholinesterase, α₂-lipoprotein(s), myoglobin, C-reactive Protein, α₂-macroglobulin, α₂-HS-glycoprotein, Zn-α₂-glycoprotein, α₂-neuramino-glycoprotein, erythropoietin, β-lipoprotein, transferrin, hemopexin, fibrinogen, plasminogen, β₂-glycoprotein I, β₂-glycoprotein II, immunoglobulins A, D, E, G, M, prothrombin, thrombin, and protein markers in cancers including, but not limited to, breast cancer, prostate cancer, melanoma, carcinoma, pancreatic cancer, liver cancer, and brain cancer.

Additional protein analytes of interest include alanine aminotransferase and aspartate aminotransferase. Alanine aminotransferase is markedly elevated when hepatitis is present in the liver. Such elevation for alanine aminotransferase may include at least about 1.25, 1.5, 1.75, 2, 2.25, 2.5, 2.75, and 3.0 times the normal levels associated with a person lacking liver damage. Aspartate aminotransferase is elevated when cellular damage occurs, such as liver damage, skeletal muscle damage, and acute myocardial infarction. Additionally, levels are elevated because of congestive heart failure, pericarditis, cirrhosis, metastatic liver disease, skeletal muscle diseases, and generalized infections such as mononucleosis. Such elevation for aspartate aminotransferase may include at least about 1.25, 1.5, 1.75, 2, 2.25, 2.5, 2.75, and 3.0 times the normal levels associated with a person lacking liver damage. Consequently, the detection of alanine aminotransferase and/or aspartate aminotransferase is of therapeutic importance.

Specific examples of peptide and protein hormone analytes include parathyroid hormone (parathromone), thyrocalcitonin, insulin, glucagon, relaxin, erythropoietin, melanotropin (melanocyte-stimulating hormone and intermedin), somatotropin (growth hormone), corticotropin (adrenocorticotropic hormone), thyrotropin, prolactin, follicle-stimulating hormone, luteinizing hormone), chorionic gonadotropin (hCG), oxytocin, and vasopressin.

Specific examples of polynucleotide analytes include DNA and RNA as well as their nucleoside and nucleotide precursors, which include ATP, NAD, FMN, adenosine, guanosine, thymidine, cytidine, and uracil with their appropriate sugar and phosphate substituents.

Specific examples of vitamin analytes include Vitamin A (i.e. retinol), B (e.g. B₁ or thiamine, B₂ or riboflavin, B₃ or niacin, B₅ or pantothenate, B₆ or pyridoxine, B₇ or biotin, B₉ or folic acid, and B₁₂), C (i.e. ascorbic acid), D (e.g. calciferol, D₂, and D₃), E (i.e. tocopherol), K, and vitamin derivatives or metabolites such as nicotinamide.

Specific examples of microorganism analytes, including infectious disease agents, include corynebacteria, pneumococci, streptococci, staphylococci, neisseriae, hemophilus influenzae, pasteurellae, brucellae, aerobic spore-forming bacilli, anaerobic spore-forming bacilli, mycobacteria, actinomycetes (fungus-like bacteria), the spirochetes, mycoplasmas, and other pathogens, such as listeria monocytogenes, erysipelothrix rhusiopathiae, streptobacillus moniliformis, donvania granulomatis, bartonella bacilliformis, rickettsiae (bacteria-like parasites), fungi, agents causing venereal diseases such as chlamydia, chancroid, granuloma inguinale, gonorrhea, syphilis, jock itch, yeast infection, herpes simplex, HPV, crab louse, scabies, trichomoniasis, and infectious diarrheal microorganisms such as camplylobacter, salmonellae, shigellae, Escherichia coli, Clostridium difficile, Giardia lamblia, Entamoeba histolytica, and organisms causing leptospirosis, nosocomial infections, staphylococcal enterotoxicosis, typhoid fever, cholera, vibrio gastroenteritis, yersinia gastroenteritis, clostridium perfringens gastroenteritis, bacillus cereus gastroenteritis, aflatoxin poisoning, amoebic dysentery, cryptosporidiosis, cyclospora diarrheal infection. Other microorganism analytes include viruses, such as herpes viruses, pox viruses, picornaviruses, myxoviruses (influenza A, B, and C, and mumps, measles, rubella, etc.), arboviruses, reoviruses, rotoviruses, noroviruses, adenoviruses, astroviruses, hepatitis, human immunodeficiency virus, and tumor viruses.

The categories of protein analytes and microorganism analytes may optionally overlap. For example, a microorganism analyte may be detected via the analysis of a protein analyte specific for the microorganism analyte. A protein analyte specific for a microorganism analyte may include an antibody specific for a microorganism analyte, or marker thereof. As a non-limiting example, for a microorganism analyte such as viral hepatitis, antibodies specific to any of viral hepatitis A, B, C, D, E, F and/or G may comprise the protein analyte. Such antibodies include, but are not limited to, immunoglobins such as IgA, IgD, IgE, and specifically IgM and/or IgG, and antibodies to surface antigens, envelope antigens, core antigens, and/or delta antigens (e.g. small and/or large). Specific examples of antigens for viral hepatitis B include hepatitis B surface antigen (HBsAg), hepatitis B envelope antigen (HBeAg), hepatitis B core antigen (HBcAg). Alternatively, a protein analyte specific for a microorganism analyte may include a protein analyte characteristically produced by the microorganism analyte. As a non-limiting example, for a microorganism analyte such as viral hepatitis, proteins specific to any of viral hepatitis A, B, C, D, E, and/or F may comprise the protein analyte. Such protein analytes include, but are not limited to, structural and/or nonstructural proteins. Specific examples of protein analytes for viral hepatitis C include, but are not limited to structural proteins such as E1 and/or E2, and/or nonstructural proteins such as NS2, NS3, NS4, NS4A, NS4B, NS5, NS5A, NS5B, and peptide portions thereof.

The above described analytes possess at least one marker recognized by at least one test reagent and/or signaling reagent. Optionally, the above described analytes may possess multiple markers recognized by the same and/or different test reagents and/or signaling reagents. It is readily envisioned that a marker may be the entire analyte and/or a portion thereof.

### Samples

An analyte of interest may be present in a wide variety of environments, and it is envisioned that a person having ordinary skill in the art will readily understand that the components and embodiments discussed above can be modified as needed to accommodate different environments of samples.

Analytes of interest may be found in a patient's physiological fluids, such as mucus, blood, serum, blood plasma, lymph, puss, urine, feces, cerebral spinal fluid, ocular lens liquid, ascites, semen, sputum, sweat, secreted oils, and preferably saliva. Samples for testing analytes may be obtained using techniques known or envisioned to provide samples of such physiological fluids. Optionally, analytes may be detected by directly contacting embodiments of the diagnostic test strips with the patient's body, such as their skin, eyes, mouth cavity regions including the tongue, tonsils, and inner lining of the mouth and throat, and the nasal cavity.

For oral fluids such as saliva, samples may be obtained by contacting an embodiment with a patient's tongue such that the tongue contacts the one or more test pads. Alternatively, salivary samples may be obtained by contacting an embodiment with the top and/or sides of a patient's tongue using a substantially back and forth motion from substantially the tip of the tongue to substantially the back of the tongue. Furthermore, salivary samples may be obtained by contacting an embodiment with the top and/or sides of a patient's tongue using a substantially side-to-side motion along the width of the tongue. Similarly, salivary samples may also be obtained by contacting an embodiment with the top and/or sides of a patient's tongue using a substantially circular motion. For each of the above described sample collection methods, the results of the analysis could then be read directly from the diagnostic test strip. Optionally, test results could be stored to a suitable memory device for recordation and later access.

### CONCLUSION

While the invention has been described with reference to the specific embodiments thereof, it should be understood by those skilled in the art that various changes may be made and equivalents may be substituted without departing from the true spirit and scope of the invention. This includes embodiments which do not provide all of the benefits and features set forth herein. In addition, many modifications may be made to adapt a particular situation, material, composition of matter, process, process step or steps, to the objective, spirit and scope of the present invention. All such modifications are intended to be within the scope of the claims appended hereto. Accordingly, the scope of the invention is defined only by reference to the appended claims.

What is claimed is:

1. A diagnostic test strip comprising a carrier strip, one or more test pads, and one or more boundary projections, wherein the one or more test pads and the one or more boundary projections are opposed on the opposite or same side of the carrier strip, and further wherein the boundary projections have an opening such that the boundary projections substantially surrounds three sides of each of the one or more test pads.

2. The diagnostic test strip of claim 1, wherein the one or more test pads and the one or more boundary projections are on the same side of the carrier strip and are substantially at one end of the carrier strip.

3. The diagnostic test strip of claim 2, wherein the opening in the one or more boundary projections faces toward the end of the carrier strip closest to the one or more test pads.

4. The diagnostic test strip of claim 2, wherein the opening in the one or more boundary projections faces away from the end of the carrier strip closest to the one or more tests pads.

5. The diagnostic test strip of claim 1, wherein there are two or more boundary projections on the same side of the carrier strip and further wherein at least one opening in a boundary projection faces away from the closest end of the carrier strip closest to the one or more tests pads and the opening in another boundary region faces toward the closest end of the carrier strip.

6. The diagnostic test strip of claim 1, wherein there are two or more boundary projections on the same side of the carrier strip and further wherein at least one each of the two or more boundary projections are substantially at the opposite ends of the carrier strip and the opening in each one faces toward the nearest end.

7. The diagnostic test strip of claim 1, wherein there are two or more boundary projections on the same side of the carrier strip further wherein at least one each of the two or more boundary projections are substantially at the opposite ends of the carrier strip and the opening in each one faces away from the nearest end.

8. The diagnostic test strip of claim 1, wherein there are two or more boundary projections on the same side of the carrier strip and further wherein at least one each of the two or more boundary projections are substantially at the opposite ends of the carrier strip and the opening in one faces away from the nearest end and the opening in the other faces toward the nearest end.

9. The diagnostic test strip of claim 1, wherein there are two or more boundary projections on the same side of the carrier strip and further wherein at least two of the two or more boundary projections are placed substantially in the middle of the carrier strip and the opening in each one faces away from each other and toward the nearest end.

10. The diagnostic test strip of claim 1, wherein there are two or more boundary projections on the same side of the carrier strip and further wherein at least two of the two or more boundary projections are placed substantially in the middle of the carrier strip and the opening in each one faces toward each other.

11. The diagnostic test strip of claim 1, wherein there are two or more boundary projections on the same side of the carrier strip and further wherein at least two of the two or more boundary projections are placed substantially in the middle of the carrier strip and the opening in each one faces the same end of the test strip.

12. The diagnostic test strip of claim 1, wherein there are two or more boundary projections on the same side of the carrier strip and further wherein at least one of the two or more boundary projections is placed substantially in the middle of the carrier strip and the other is placed substantially at one end of the test strip and the opening in each one faces away from each other and toward the nearest end.

13. The diagnostic test strip of claim 1, wherein there are two or more boundary projections on the same side of the carrier strip and further wherein at least one of the two or more boundary projections is placed substantially in the middle of the carrier strip and the other is placed substantially at one end of the test strip and the opening in each one faces toward the same end of the carrier strip.

14. The diagnostic test strip of claim 1, wherein there are two or more boundary projections on the same side of the carrier strip and further wherein at least one of the two or more boundary projections is placed substantially in the middle of the carrier strip and the other is placed substantially at one end of the test strip and the opening in each one faces toward the same side of the carrier strip.

15. The diagnostic test strip of claim 1, wherein there are two or more boundary projections on the same side of the carrier strip and further wherein at least one of the two or more boundary projections is placed substantially in the middle of the carrier strip and the other is placed substantially at one end of the test strip and the opening in each one faces substantially toward opposite sides of the carrier strip.

16. The diagnostic test strip of claim 1, wherein the sides of the one or more boundary projections slope downward towards the opening of the boundary projection.

17. The diagnostic test strip of claim 1, wherein the sides of the one or more boundary projections extend substantially for the entire length of the one or more test pads.

18. The diagnostic test strip of claim 1, wherein the sides of the one or more boundary projections extend partially along the length of the one or more test pads inside and closest to the end the boundary projection.

19. The diagnostic test strip of claim 1, wherein there are two test pads.

20. The diagnostic test strip of claim 1, wherein there is one test pad.

21. The diagnostic test strip of claim 1, wherein the one or more test pads each contain a test reagent.

22. The diagnostic test strip of claim 1, wherein the one or more test pads each contain a different test reagent.

23. The diagnostic test strip of claim 1, wherein the one or more test pads are on the same side of the carrier strip and contain two test reagents on different regions of the test pad.

24. The diagnostic test strip of claim 23, wherein the test reagents are different.

25. The diagnostic test strip of claim 23, wherein the two test reagents are arranged in a pattern to give a signal to the user.

26. The diagnostic test strip of claim 1, wherein the carrier strip is porous.

27. The diagnostic test strip of claim 1, wherein the carrier strip is non-porous.

28. The diagnostic test strip of claim 1, wherein the one or more test pads are porous.

29. The diagnostic test strip of claim 1, wherein the one or more test pads are non-porous.

30. The diagnostic test strip of claim 1, wherein there are at least two or more test pads each with a different test reagent and each reagent tests for a different marker on the same analyte.

31. The diagnostic test strip of claim 1, wherein at least one test pad further contains a signaling reagent.

32. The diagnostic test strip of claim 1, further comprising a substantially non-porous handle attached on one end or side of the carrier strip.

33. The diagnostic test strip of claim 1, wherein the carrier strip is substantially square shaped.

34. The diagnostic test strip of claim 1, wherein the carrier strip is substantially circularly shaped.

35. The diagnostic test strip of claim 34, wherein there are at least two boundary projections on the same side of the carrier strip and the projections are placed substantially opposite on substantially the edge of the circularly shaped carrier strip.

36. The diagnostic test strip of claim 35, wherein the openings in the boundary projections face toward the edge of the carrier strip.

37. The diagnostic test strip of claim 35, wherein the openings in the boundary projections face away from the edge of the carrier strip.

38. The diagnostic strip of claim 35, further wherein there is at least one boundary projection in the substantially center of the carrier strip.

39. The diagnostic test strip of claim 35, further wherein there are at least two boundary projections on the same side of the carrier strip each substantially in the center of the carrier strip and the openings in each one face substantially away from each other.

40. The diagnostic test strip of claim 34, wherein there are three or more boundary projections on the same side of the carrier strip placed substantially at the edge of the carrier strip and substantially evenly spaced from each other.

41. The diagnostic test strip of claim 40, wherein the openings in the boundary projections face substantially toward the edge of the carrier strip.

42. The diagnostic test strip of claim 40, wherein the openings in the boundary projections face substantially away from the edge of the carrier strip.

43. The diagnostic test strip of claim 40, wherein the openings in the boundary projections alternate in facing substantially to and away from the edge of the carrier strip.

44. The diagnostic test strip of claim 33, comprising two or more concentric circles of multiple boundary projections on the same side of the carrier strip substantially evenly spaced from each other.

45. The diagnostic test strip of claim 1, wherein there are two or more boundary projections and at least one is on the opposite side of the others.

46. The diagnostic test strip of claim 1, wherein the carrier strip is substantially oval shaped.

47. A method for detecting one or more analytes in a patient sample, comprising:
a) contacting the test strip of claim 1 with a patient's tongue so that the patient's tongue contacts the one or more test pads; and
b) reading the results from the test strip.

48. The method of claim 47, further comprising contacting the test strip with one or more signaling reagents so that the one or more reagents contact the one or more test pads.

49. The method of claim 47, wherein the test strip is contacted with the top and the sides of the patient's tongue in a substantially back and forth motion from substantially the tongue tip to substantially the back of the tongue.

50. The method of claim 47, wherein the test strip is contacted with the top and the sides of the patient's tongue in a substantially side-to-side motion along the width of the tongue.

51. The method of claim 47, wherein the test strip is contacted with the top and the sides of the patient's tongue in a substantially circular motion.
